SEQUENCE LISTING (12 sequences)

SEQ ID NO: 1 - protein, 556 aa, Homo sapiens [sequence omitted]

SEQ ID NO: 2 - protein, 16 aa, synthetic construct
RSSKSLQNVNGNTYLY

SEQ ID NO: 3 - protein, 7 aa, synthetic construct
RMSNLNS

SEQ ID NO: 4 - protein, 9 aa, synthetic construct
MQHLEYPIT

SEQ ID NO: 5 - protein, 5 aa, synthetic construct
SYVMH

SEQ ID NO: 6 - protein, 14 aa, synthetic construct
WIGYINPYNDGTKY

SEQ ID NO: 7 - protein, 12 aa, synthetic construct
GTYYYGTRVFDY

SEQ ID NO: 8 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 12 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

(19) United States
(12) Patent Application Publication
(10) Pub. No.: US 2025/0230238 A1
(43) Pub. Date: Jul. 17, 2025

(54) METHODS AND COMPOSITIONS FOR TREATING SYSTEMIC LUPUS ERYTHEMATOUS (SLE)

(71) Applicant: Zenas BioPharma, Inc., Waltham, MA (US)

(72) Inventors: Stephen Wax, Auburndale, MA (US); Allen Poma, Somerville, MA (US); Ginger Pocock, San Antonio, TX (US)

(21) Appl. No.: 19/029,006

(22) Filed: Jan. 17, 2025

Related U.S. Application Data
(60) Provisional application No. 63/621,961, filed on Jan. 17, 2024.

(51) Int. Cl. C07K 16/28

(52) U.S. Cl. CPC C07K 16/2803; C07K 16/283; C07K 2317/24; C07K 2317/31; C07K 2317/76

(57) ABSTRACT

The present invention provides methods of treating and improving the symptoms of system lupus crythematous using obexclimab.

### CROSS REFERENCE TO RELATED APPLICATIONS

The present application claims benefit of and priority to U.S. Provisional Patent Application No. 63/621,961, filed on Jan. 17, 2024, which is hereby incorporated by reference in its entirety.

### SEQUENCE LISTING

The present application contains a Sequence Listing which has been submitted electronically in XML file format and is hereby incorporated by reference in its entirety. Said XML copy, created Jan. 15, 2025, is named ZEN-021US1_SL and is 13,153 bytes.

### BACKGROUND

Systemic lupus erythematous (SLE), the most common form of lupus, is a complex, chronic systemic autoimmune disease that may affect multiple organs. At least 1.5 million Americans are afflicted by lupus and more than 16,000 new cases are reported annually. It is estimated that five million people throughout the world suffer from some form of lupus, of which 70% suffer from the most common form, SLE. Lupus affects primarily women of childbearing age (15 to 44 years). However, men, children, and teenagers can also develop lupus. SLE is typically a disease of young women ages 15-45, with a 10-fold greater incidence in women than in men. There are some ethnic differences, with greater prevalence and severity of disease in persons of African, Hispanic, Asian, and Native American descent. While the risk of mortality has decreased to 5-10% at 10 years, human subjects still die of active disease, infection, cardiovascular causes, and treatment associated effects. Despite improved survival, it is estimated that only 15% of human subjects have good to excellent disease control sustained for 1 year.

SLE is currently incurable. The goals of treatment are to reduce inflammation and damage to the organs and to prevent or reverse disease exacerbations. Therapy is tailored to the organ systems involved and the amount of inflammation, but most of the agents used are non-specific immunosuppressants that are frequently used in combination. For milder forms of SLE involving skin and/or joints, topical or low dose oral steroids, hydroxychloroquine, non-steroidal anti-inflammatory drugs (NSAIDs) and/or methotrexate are often the mainstay of therapy. Involvement of other organ systems often warrants higher doses of oral steroids together with agents such as azathioprine, mycophenolate mofetil, anifrolumab, or belimumab. Aggressive treatment is warranted when vital organs are involved to prevent organ damage or failure. High dose oral or IV corticosteroids with cyclophosphamide, azathioprine, or mycophenolate mofetil may be used for organ threatening disease in the kidneys, CVS, and hematopoietic systems. Many of the therapeutics are less than optimal therapies particularly for young women because of their long-term safety profiles. For example, long-term corticosteroid use can lead to hypertension, diabetes, osteoporosis, and infection risk, whereas cyclophosphamide may lead to sterility and bladder cancer. Furthermore, due to the unpredictable course of this disease and the involvement of many organs, treating SLE has been and continues to be challenging for physicians as the current standard of care is to prescribe treatments empirically. Therefore, there is a need for more targeted agents to control the disease long-term.

### SUMMARY

Among other things, the present disclosure provides improved methods for treating systemic lupus erythematous (SLE) using obexelimab.

In one aspect, the present invention provides a method of treating systemic lupus erythematous (SLE), comprising administering obexelimab subcutaneously to a human patient with SLE at a dose of 250 mg once a week or an equivalent dosing regimen thereof.

In some embodiments, the patient meets the 2019 European League Against Rheumatism (EULAR) and the American College of Rheumatology (ACR) Classification Criteria for SLE. In some embodiments, the patient meets the 2019 European League Against Rheumatism (EULAR) and the American College of Rheumatology (ACR) Classification Criteria for SLE by accumulating ≥10 points of the criteria.

In some embodiments, the patient: (a) is positive for antinuclear antibodies (ANA) at a titer of ≥1:80; (b) is positive for anti-Smith antibodies; or (c) is positive for anti-dsDNA antibodies; prior to the administration of obexelimab.

In some embodiments, the patient has active SLE. In some embodiments, the active SLE: (a) has a disease activity score greater than or equal to 6 according to the hybrid Systemic Lupus Erythematosus Disease Activity Index (hSLEDAI); and/or, (b) has a British Isles Lupus Assessment Group (BILAG)-2004 grade A or B in greater than or equal to I organ system. In some embodiments, the active SLE: (a) has a disease activity score greater than or equal to 6 according to the hybrid Systemic Lupus Erythematosus Disease Activity Index (hSLEDAI); and, (b) has a British Isles Lupus Assessment Group (BILAG)-2004 grade A or B in greater than or equal to I organ system. In some embodiments, the disease activity score is 6 to 9 according to hSLEDAI. In some embodiments, the disease activity score is greater than or equal to 10 according to hSLEDAI.

In some embodiments, the patient has previously received or is further administered a standard of care therapy. In some embodiments, the standard of care therapy comprises administering an immunosuppressant. In some embodiments, the immunosuppressant comprises mycophenolate mofetil, mycophenolate sodium, azathioprine, 6-mercaptopurine, methotrexate, cyclosporine, tacrolimus, or voclosporin. In some embodiments, the standard of care therapy comprises administering a corticosteroid. In some embodiments, the corticosteroid comprises a glucocorticoid steroid. In some embodiments, the glucocorticoid steroid comprises prednisone. In some embodiments, the patient is administered up to about 20 mg/day prednisone-equivalent. In some embodiments, the corticosteroid is tapered to a dose of less than or equal to 5 mg/day prednisone-equivalent within 12 weeks of administered of obexelimab. In some embodiments, the standard of care therapy comprises administering an antimalarial. In some embodiments, the antimalarial comprises hydroxychloroquine, quinacrine, or chloroquine.

In some embodiments, the patient achieves one or more of the following: (a) a response according to the BILAG-Based Composite Lupus Assessment (BICLA); (b) a response according to the Systemic Lupus Erythematosus Responder Index 4 (SRI-4), Systemic Lupus Erythematosus Responder Index 6 (SRI-6), or Systemic Lupus Erythematosus Responder Index 8 (SRI-8); (c) Lupus Low Disease Activity State (LLDAS); (d) a ≥50% improvement from baseline in Cutaneous Lupus Erythematosus Disease Area and Severity Index Activity Score (CLASI-50 response); (e) a ≥50% decrease in joint count (Joint-50 response) as compared to baseline; or (f) a change from baseline in Functional Assessment of Chronic Illness Therapy (FACIT) Fatigue Score.

In some embodiments, obexelimab is administered in a liquid formulation comprising 125 mg/mL obexelimab, 2.35 mg/mL sodium acetate trihydrate, 0.17 mg/mL acetic acid, 30 mg/mL L-proline, 0.1 mg/mL polysorbate 80 at pH 5.5. In some embodiments, obexelimab is administered as 2×1 mL injections or 1×2 mL injection. In some embodiments, obexelimab is administered using a prefilled syringe or autoinjector.

### BRIEF DESCRIPTION OF THE DRAWINGS

Drawings are for illustration purposes and not for limitation.

FIG. 1 illustrates an exemplary clinical study design for treating SLE.

### DETAILED DESCRIPTION

The present invention provides, among other things, methods of treating systemic lupus erythematosus (SLE) in a subject in need thereof comprising administering obexelimab at a therapeutically effective dose and administration interval for a treatment period sufficient to improve, stabilize, or reduce one or more symptoms.

Various aspects of the invention are described in detail in the following sections. The use of sections is not meant to limit the invention. Each section can apply to any aspect of the invention. In this application, the use of “or” means “and/or” unless stated otherwise.

### Definitions

Described herein are several definitions. Such definitions are meant to encompass grammatical equivalents.

Antibody: The term “antibody” herein is meant to include a protein consisting of one or more polypeptides substantially encoded by all or part of the recognized immunoglobulin genes. The recognized immunoglobulin genes, for example in humans, include the kappa (K), lambda (l), and heavy chain genetic loci, which together comprise the myriad variable region genes, and the constant region genes mu (u), delta (d), gamma (γ), sigma(s), and alpha (a) which encode the IgD, IgG (lgG1, lgG2, lgG3, and lgG4), IgE, and IgA (lgA1 and lgA2) isotypes respectively. Antibody herein is meant to include full length antibodies and antibody fragments, and may refer to a natural antibody from any organism, an engineered antibody, or an antibody generated recombinantly for experimental, therapeutic, or other purposes.

Equivalent dosing regimen: As used herein, the term “equivalent dosing regimen” or other grammatical equivalents refer to an alternative dosing regimen that delivers a substantially equivalent amount of a therapeutic agent (e.g., an antibody, a protein or small molecular compound drug) over a comparable dosing period or otherwise results in similar pharmacodynamic (PD) and/or pharmacokinetic (PK) response in vivo. As non-limiting examples, an equivalent dosing regimen of 250 mg once a week can be 125 mg twice a week, or 500 mg once every other week, or 1000 mg once a month. In some embodiments, an equivalent dosing regimen may achieve a similar area under the curve (AUC), C_{average}, Cₘᵢₙ, and/or Emax over a comparable dosing period.

Fc or Fc region: The terms “Fc” or “Fc region,” as used herein is meant the polypeptide comprising the constant region of an antibody excluding the first constant region immunoglobulin domain and in some cases, part of the hinge. Thus, Fc may refer to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cgamma2 and Cgamma3 (Og2 and C 3) and the hinge between Cgamma1 (Ogî) and Cgamma2 (Cy2). Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an Fc polypeptide, as described below.

Fc gamma receptor, or FcγR: The terms “Fc gamma receptor” or “FcγR” as used herein is meant any member of the family of proteins that bind the IgG antibody Fc region and are substantially encoded by the FcγR genes. In humans this family includes but is not limited to FcγRI (CD64), including isoforms FcγRIa, FcγRIb, and FcγRIc; FcγRII (CD32), including isoforms FcγRIla (including allotypes H131 and R131), FcγRIIb (including FcγRllb-1 and FcγRllb-2), and FcγRIIc; and FcγRI 11 (CD16), including isoforms FcγRI lla (including allotypes V1 58 and F158) and FcγRIIIb (including allotypes FcγRlllb-NA1 and FcγRI llb-NA2) (Jefferis et al., 2002, Immunol Lett 82:57-65, incorporated entirely by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγR 111 (CD16), and FcγRIII-2 (CD16-2), as well as any undiscovered mouse FcγRs or FcγR isoforms or allotypes.

Modification: The term “modification” herein is meant an alteration in the physical, chemical, or sequence properties of a protein, polypeptide, antibody, or immunoglobulin. Modifications described herein include amino acid modifications (including amino acid substitutions) and glycoform modifications.

Treating: The term “treating” as used herein is meant to encompass improving, ameliorating, or delaying the onset of one or more symptoms of a disease or disorder.

### Systemic Lupus Erythematous (SLE)

SLE is characterized most notably by unpredictable flares in joints, skin, kidneys, and other vital organs that cause progressive organ damage. The prognosis of SLE is highly variable in individual patients, often waxing and waning throughout their lifetime. The natural history of SLE ranges from relatively benign disease to rapidly progressive and even fatal disease. Comorbidities, such as infections, malignancies, hypertension, lipid disorders, and diabetes increase the risk of disability and death in patients with SLE. Organ systems commonly affected by SLE include the skin, musculoskeletal system, central nervous system (CNS), kidneys, gastrointestinal (GI) system, mucous membranes, heart, hematologic system, and lungs, with specific organ involvement defining subsets of the disease (e.g., lupus nephritis).

The American College of Rheumatology (ACR)/European League Against Rheumatism (EULAR) Classification Criteria for SLE has been developed and validated by a panel of 200 international experts in SLE, using a cohort of almost 4000 patients. To fulfill ACR/EULAR Classification Criteria for SLE, patients must have positive ANA at least once as obligatory entry criterion; followed by additive weighted criteria grouped in 7 clinical (constitutional, hematological, neuropsychiatric, mucocutaneous, serosal, musculoskeletal, renal) and 3 immunological (antiphospholipid antibodies, complement proteins, SLE-specific antibodies) domains, and weighted from 2 to 10. Patients much achieve a score of ≥10 to be classified.

Current treatments for SLE include aspirin, corticosteroids, hydroxychloroquine, voclosporin (United States Food and Drug Administration [FDA] approved for lupus nephritis in 2021), belimumab (FDA approved for SLE in 2011), and anifrolumab (FDA approved for SLE in 2021). These limited options are superimposed on an array of unapproved immune modulators which dominate standard care but are poorly studied, not universally effective, and can cause significant toxicity. Effect sizes in SLE clinical studies have been modest (12% to 17%) for the approved therapies. Few clinically validated biomarkers exist to guide the selection or optimal administration of treatments, and thus current standard care is to prescribe treatments empirically.

B cells play a critical role in SLE pathogenesis, from producing autoantibodies to abnormal regulation of immune responses. Moreover, SLE is an autoimmune disease characterized by B cell dysfunction, the production of autoantibodies toward cellular and nuclear components, and multiorgan damage caused by immune complex deposition and inflammation within affected tissues. Increased B cell activity and survival mediated through B cell receptor (BCR) abnormalities is a classic feature of SLE. BCR signals are impacted by several regulators, including CD19, a B cell-restricted surface protein, and CD32b, the FcγRIIb. Mutations in the CD32b gene in humans are associated with an increased likelihood of SLE, and reduced expression of CD32b is apparent in B cells from SLE patients. Activation of immunoreceptor tyrosine-based inhibitory motif of FcγRIIb is known to effectively dampen BCR signaling and decrease B cell responses to activating signals, which may play a pivotal role in suppressing autoimmunity in both murine lupus models and human lupus studies. These data provide a rationale for targeting CD19 and enhancing the functionality of FcγRIIb as a treatment for SLE.

The pathogenesis of SLE includes abnormal B cell activation, leading to the production of pathogenic autoantibodies. Obexelimab is designed to inhibit the activity of the broad range of CD19 positive cells in the B cell lineage including a subset of plasma cells (which are the cells that produce the majority of antibodies and autoantibodies).

### Obexelimab

In some embodiments, obexelimab is used to treat a human patient with SLE. In one aspect, the present invention provides a method of administering obexelimab for the treatment of SLE. Obexelimab is a monoclonal antibody specific for CD19 comprising: a light chain comprising a variable region having:
a CDR1 comprising RSSKSLQNVNGNTYLY (SEQ ID NO: 2),
a CDR2 comprising RMSNLNS (SEQ ID NO: 3), and
a CDR3 comprising MQHLEYPIT (SEQ ID NO: 4); and
   a heavy chain comprising a variable region having
a CDR1 comprising SYVMH (SEQ ID NO: 5),
a CDR2 comprising WIGYINPYNDGTKY (SEQ ID NO: 6), and
a CDR3 comprising GTYYYGTRVFDY (SEQ ID NO: 7),
   wherein the heavy chain comprises amino acid substitutions in the Fc region S267E and L328F as compared to SEQ ID NO: 8:

(SEQ ID NO: 8)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK,

wherein the numbering is according to the EU index, as in Kabat.

In some embodiments, obexelimab comprises: a light chain comprising amino acid sequence:

(SEQ ID NO: 9)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS;

and heavy chain comprising an amino acid sequence:

(SEQ ID NO: 10)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVEHEDPEVKFNWYVDGVEVHNAKTKPREEQY
NSTYRVVSVLTVLHQDWLNGKEYKCKVSNKAFPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K.

TABLE 1
Sequence of Heavy chain and Light chain amino acid
sequence of obexelimab.

| Heavy Chain | Light Chain |
|---|---|
| EVQLVESGGGLVKPGGSLKLSCAASGY | DIVMTQSPATLSLSPGERATLSCRSSKSLQN |
| TFTSYVMHWVRQAPGKGLEWIGYINPY | VNGNTYLYWFQQKPGQSPQLLIYRMSNLNS |
| NDGTKYNEKFQGRVTISSDKSISTAYME | GVPDRFSGSGSGTEFTLTISSLEPEDFAVYYC |
| LSSLRSEDTAMYYCARGTYYYGTRVFD | MQHLEYPITFGAGTKLEIKRTVAAPSVFIFPP |
| YWGQGTLVTVSSASTKGPSVFPLAPSSK | SDEQLKSGTASVVCLLNNFYPREAKVQWK |
| STSGGTAALGCLVKDYFPEPVTVSWNS | VDNALQSGNSQESVTEQDSKDSTYSLSSTLT |
| GALTSGVHTFPAVLQSSGLYSLSSVVTV | LSKADYEKHKVYACEVTHQGLSSPVTKSFN |
| PSSSLGTQTYICNVNHKPSNTKVDKKVE | RGEC (SEQ ID NO:9) |
| PKSCDKTHTCPPCPAPELLGGPSVFLFPP | |
| KPKDTLMISRTPEVTCVVVDVEHEDPE | |
| VKFNWYVDGVEVHNAKTKPREEQYNS | |
| TYRVVSVLTVLHQDWLNGKEYKCKVS | |
| NKAFPAPIEKTISKAKGQPREPQVYTLPP | |
| SREEMTKNQVSLTCLVKGFYPSDIAVE | |
| WESNGQPENNYKTTPPVLDSDGSFFLY | |
| SKLTVDKSRWQQGNVFSCSVMHEALH | |
| NHYTQKSLSLSPGK (SEQ ID NO: 10) | |

Variable region

| | |
|---|---|
| EVQLVESGGGLVKPGGSLKLSCAASGY | DIVMTQSPATLSLSPGERATLSCRSSKSLQN |
| TFTSYVMHWVRQAPGKGLEWIGYINPY | VNGNTYLYWFQQKPGQSPQLLIYRMSNLNS |
| NDGTKYNEKFQGRVTISSDKSISTAYME | GVPDRFSGSGSGTEFTLTISSLEPEDFAVYYC |
| LSSLRSEDTAMYYCARGTYYYGTRVFD | MQHLEYPITFGAGTKLEIK (SEQ ID NO: 11) |
| YWGQGTLVTVSS (SEQ ID NO: 12) | |

In some embodiments, obexelimab comprises a light chain variable region and a heavy chain variable region as given by Table 1.

Obexelimab works by exploiting the regulation of B-cell receptor (BCR) signaling by FcγRIIB. Obexelimab binds CD19 of the BCR complex and its Fc is engineered to increase its affinity for the inhibitory FcγRIIB. Since CD19 is associated with the BCR, Obexelimab tethering of CD19 to FcγRIIB on the same cell poises the BCR complex for inhibition upon antigen-induced BCR aggregation. Obexelimab capitalizes upon the natural inhibitory mechanism of FcγRIIb, the only Fc receptor expressed by B cells, which acts as a negative regulator in conditions of antigen excess and immune complex formation (Chu et al., 2014). Obexelimab may also have an improved safety profile compared to B cell depleting antibodies as it may not mediate B cell killing.

### Variants

In some embodiments, a variant of obexelimab is an immunoglobulin specific for CD19 comprises: a light chain comprising a variable region having a CDR1 comprising RSSKSLQNVNGNTYLY (SEQ ID NO: 2), a CDR2 comprising RMSNLNS (SEQ ID NO: 3), and a CDR3 comprising MQHLEYPIT (SEQ ID NO: 4); and a heavy chain comprising a variable region having a CDR1 comprising SYVMH (SEQ ID NO: 5), a CDR2 comprising WIGYINPYNDGTKY (SEQ ID NO: 6), and a CDR3 comprising GTYYYGTRVFDY (SEQ ID NO: 7), wherein the heavy chain comprises amino acid substitutions in the Fc region S267E and L328F as compared to:

(SEQ ID NO: 8)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

wherein the numbering is according to the EU index, as in Kabat.

In some embodiments, a variant of obexelimab comprises a heavy chain variable region (VH) and/or a light chain variable region (VL), comprising a CDR1, a CDR2, and a CDR3, each of which differs by no more than 1, 2, 3, 4 or 5 amino acid residues from each of RSSKSLQNVNGNTYLY (SEQ ID NO: 2), RMSNLNS (SEQ ID NO: 3), MQHLEYPIT (SEQ ID NO: 4), SYVMH (SEQ ID NO: 5), WIGYINPYNDGTKY (SEQ ID NO: 6) and/or GTYYYGTRVFDY (SEQ ID NO: 7).

In some embodiments, the variant of obexelimab comprises: a light chain variable region comprising an amino acid sequence that is at least 90%, 91%, 92%, 93%, 94%, 95%, 96%, 97%, 98% or 99% identical to the light chain variable region identified in Table 1. In some embodiments, the variant of obexelimab comprises: a heavy chain variable region comprising an amino acid sequence that is at least 90%, 91%, 92%, 93%, 94%, 95%, 96%, 97%, 98% or 99% identical to the heavy chain variable region as identified in Table 1.

In some embodiments, the variant of obexelimab comprises: a light chain variable region comprising an amino acid sequence that is at least 90%, 91%, 92%, 93%, 94%, 95%, 96%, 97%, 98% or 99% identical to the light chain variable region identified in Table 1 and a heavy chain variable region comprising an amino acid sequence that is at least 90%, 91%, 92%, 93%, 94%, 95%, 96%, 97%, 98% or 99% identical to the heavy chain variable region as identified in Table 1 and further comprises amino acid substitutions in the Fc region S267E and L328F as compared to SEQ ID NO: 8, wherein the numbering is according to the EU index.

In some embodiments, the variant of obexelimab comprises: a light chain comprising an amino acid sequence 70%, 75%, 80%, 85%, 90%, 95%, 98% or 99% identical to the light chain as identified in Table 1. In some embodiments, the variant of obexelimab comprises: a heavy chain comprising an amino acid sequence 70%, 75%, 80%, 85%, 90%, 95%, 98% or 99% identical to the heavy chain as identified in Table 1.

In some embodiments, the variant of obexelimab comprises: a light chain comprising an amino acid sequence that is at least 90%, 91%, 92%, 93%, 94%, 95%, 96%, 97%, 98% or 99% identical to the light chain sequence as identified in Table 1 and a heavy chain comprising an amino acid sequence that is at least 90%, 91%, 92%, 93%, 94%, 95%, 96%, 97%, 98% or 99% identical to the heavy chain sequence as identified in Table 1, and the heavy chain of the variant comprises amino acid substitutions in the Fc region S267E and L328F as compared to SEQ ID NO: 8 wherein the numbering is according to the EU index, as in Kabat.

In some embodiments, a suitable variant of obexelimab binds to the same epitope on human CD19, as an antibody comprising a light chain and a heavy chain as identified in Table 1. Epitope binding may be determined by a method known in the art.

In some embodiments, a suitable variant of obexelimab competes for binding to human CD19, as an antibody comprising a light chain and heavy chain as identified in Table 1, under a binning assay known in the art. As used herein, a binning assay refers to any method to regionally map the epitope to which the antibody binds. Standard methods for such antibody characterization, also known as epitope binning, typically involve surface plasmon resonance (SPR) technology. Using SPR, monoclonal antibody candidates are screened pairwise for binding to a target protein. Other standard methods involve ELISA-based screens and may require synthesis of sets of overlapping peptides corresponding to the protein of interest.

In some embodiments, the human CD19 comprises an amino acid sequence of:

(SEQ ID NO: 1)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR.

### Fc Receptor Binding Properties

Anti-CD19 antibodies (e.g., obexelimab) disclosed herein comprise an Fc variant that has enhanced Fc binding to the inhibitory Fcγ receptor Iib (FcγRIIb). FcγRIIb, the only FcR on B cells, serves as an antibody-sensing down-regulator of humoral immunity that is naturally engaged by immune complexes. When sufficient antibody is raised against a given antigen, specific immune complexes form and co-engage FcγRIIb and the B cell receptor (BCR) with high avidity, selectively suppressing only B cells recognizing cognate antigen. In addition, FcγRIIb regulates the activity of other B cell stimulators including interleukin (IL)-4, LPS, and BAFF that amplify BCR-driven proliferation and differentiation. By simultaneously binding CD19 and FcγRIIb, obexelimab (and variants described herein) mimics the action of antigen-antibody complexes and down-regulates B cell activity.

The Fc variants disclosed herein may be optimized for a variety of Fc receptor binding properties. An Fc variant that is engineered or predicted to display one or more optimized properties is herein referred to as an “optimized Fc variant.” Properties that may be optimized include but are not limited to enhanced or reduced affinity for an FcγR. In one embodiment, the Fc variants disclosed herein are optimized to possess enhanced affinity for an inhibitory receptor FcγRIlb. In other embodiments, immunoglobulins disclosed herein provide enhanced affinity for FcγRIlb, yet reduced affinity for one or more activating FcγRs, including for example FcγRI, FcγRIla, FcγRIIIa, and/or FcγRIIIb. The FcγR receptors may be expressed on cells from any organism, including but not limited to human, cynomolgus monkeys, and mice. The Fc variants disclosed herein may be optimized to possess enhanced affinity for human FcγRIlb.

An Fc variant comprises one or more amino acid modifications relative to a parent Fc polypeptide, wherein the amino acid modification(s) provide one or more optimized properties. An Fc variant disclosed herein differs in amino acid sequence from its parent by virtue of at least one amino acid modification. Thus, Fc variants disclosed herein have at least one amino acid modification compared to the parent. Alternatively, the Fc variants disclosed herein may have more than one amino acid modification as compared to the parent, for example from about two to fifty amino acid modifications, e.g., from about two to ten amino acid modifications, from about two to about five amino acid modifications, etc. compared to the parent. Thus, the sequences of the Fc variants and those of the parent Fc polypeptide are substantially homologous. For example, the variant Fc variant sequences herein will possess about 80% homology with the parent Fc variant sequence, e.g., at least about 90% homology, at least about 95% homology, at least about 98% homology, at least about 99% homology, etc. Modifications disclosed herein include amino acid modifications, including insertions, deletions, and substitutions. Modifications disclosed herein also include glycoform modifications.

Modifications may be made genetically using molecular biology or may be made enzymatically or chemically.

Fc variants disclosed herein are defined according to the amino acid modifications that compose them. Thus, for example, S267E is an Fc variant with the substitution S267E relative to the parent Fc polypeptide. Likewise, S267E/L328F defines an Fc variant with the substitutions S267E and L328F relative to the parent Fc polypeptide. The identity of the WT amino acid may be unspecified, in which case the aforementioned variant is referred to as 267E/328F. It is noted that the order in which substitutions are provided is arbitrary, that is to say that, for example, 267E/328F is the same Fc variant as 328F/267E, and so on. Unless otherwise noted, positions discussed herein are numbered according to the EU index as described in Kabat (Kabat et al., 1991, Sequences of Proteins of Immunological Interest, 5^{th} Ed., United States Public Health Service, National Institutes of Health, Bethesda, hereby entirely incorporated by reference). In brief, EU is the name of the first antibody molecule whose entire amino acid sequence was determined (Edelman et al., 1969, Proc Natl Acad Sci USA 63:78-85, hereby entirely incorporated by reference), and its amino acid sequence has become the standard numbering scheme for heavy chain constant regions. The EU protein has become the standard reference for defining numbering. Kabat et al. lists the EU sequence in a set of indices aligning it with other antibody sequences, serving as a necessary tool for aligning antibodies to the EU numbering scheme. Thus, as appreciated by those of skill in the art, the standard way of referencing the EU numbering is to refer to Kabat et al.'s alignment of sequences, because it puts EU in context with antibodies of other variable domain lengths. As such, as used herein, “the EU index as in Kabat” or “numbering is according to the EU index, as in Kabat” refers to the numbering of the EU antibody as described in Kabat.

In certain embodiments, the Fc variants disclosed herein are based on human IgG sequences, and thus human IgG sequences are used as the “base” sequences against which other sequences are compared, including but not limited to sequences from other organisms, for example rodent and primate sequences. Immunoglobulins may also comprise sequences from other immunoglobulin classes such as IgA, IgE, and the like. It is contemplated that, although the Fc variants disclosed herein are engineered in the context of one parent IgG, the variants may be engineered in or “transferred” to the context of another, second parent IgG. This is done by determining the “equivalent” or “corresponding” residues and substitutions between the first and second IgG, typically based on sequence or structural homology between the sequences of the first and second IgGs. In order to establish homology, the amino acid sequence of a first IgG outlined herein is directly compared to the sequence of a second IgG. After aligning the sequences, using one or more of the homology alignment programs known in the art (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first immunoglobulin are defined. Alignment of conserved residues may conserve 100% of such residues. However, alignment of greater than 75% or as little as 50% of conserved residues is also adequate to define equivalent residues. Equivalent residues may also be defined by determining structural homology between a first and second IgG that is at the level of tertiary structure for IgGs whose structures have been determined. In this case, equivalent residues are defined as those for which the atomic coordinates of two or more of the main chain atoms of a particular amino acid residue of the parent or precursor (N on N, CA on CA, Con C and O on O) are within about 0.13 nm, after alignment. In another embodiment, equivalent residues are within about 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent IgG in which the IgGs are made, what is meant to be conveyed is that the Fc variants discovered as disclosed herein may be engineered into any second parent IgG that has significant sequence or structural homology with the Fc variant. Thus, for example, if a variant antibody is generated wherein the parent antibody is human lgG1, by using the methods described above or other methods for determining equivalent residues, the variant antibody may be engineered in another lgG1 parent antibody that binds a different antigen, a human lgG2 parent antibody, a human IgA parent antibody, a mouse lgG2a or lgG2b parent antibody, and the like. Again, as described above, the context of the parent Fc variant does not affect the ability to transfer the Fc variants disclosed herein to other parent IgGs.

The term “greater affinity” or “improved affinity” or “enhanced affinity” or “better affinity” than a parent Fc polypeptide, as used herein is meant that an Fc variant binds to an Fc receptor with a significantly higher equilibrium constant of association (KA or Ka) or lower equilibrium constant of dissociation (KD or Kd) than the parent Fc polypeptide when the amounts of variant and parent polypeptide in the binding assay are essentially the same. For example, the Fc variant with improved Fc receptor binding affinity may display from about 5 fold to about 1000 fold, e.g. from about 10 fold to about 500 fold improvement in Fc receptor binding affinity compared to the parent Fc polypeptide, where Fc receptor binding affinity is determined, for example, by the binding methods disclosed herein, including but not limited to Biacore, by one skilled in the art. Accordingly, by “reduced affinity” as compared to a parent Fc polypeptide as used herein is meant that an Fc variant binds an Fc receptor with significantly lower KA or higher KD than the parent Fc polypeptide. Greater or reduced affinity can also be defined relative to an absolute level of affinity. For example, according to the data herein, WT (native) lgG1 binds FcγRIlb with an affinity of about 1.5 mM, or about 1500 nM. Furthermore, some Fc variants described herein bind FcγRIIb with an affinity about 10-fold greater to WT lgG1. As disclosed herein, greater or enhanced affinity means having a KD lower than about 100 nM, for example between about 10 nM-about 100 nM, between about 1-about 100 nM, or less than about 1 nM.

In one embodiment, the Fc variants provide selectively enhanced affinity to FcγRIIb relative to one or more activating receptors. Selectively enhanced affinity means either that the Fc variant has improved affinity for FcγRIIb relative to the activating receptor(s) as compared to the parent Fc polypeptide but has reduced affinity for the activating receptor(s) as compared to the parent Fc polypeptide, or it means that the Fc variant has improved affinity for both FcγRIIb and activating receptor(s) as compared to the parent Fc polypeptide, however the improvement in affinity is greater for FcγRIIb than it is for the activating receptor(s). In alternate embodiments, the Fc variants reduce or ablate binding to one or more activating FcγRs, reduce or ablate binding to one or more complement proteins, reduce or ablate one or more FcγR-mediated effector functions, and/or reduce or ablate one or more complement-mediated effector functions.

The presence of different polymorphic forms of FcγRs provides yet another parameter that impacts the therapeutic utility of the Fc variants disclosed herein. Whereas the specificity and selectivity of a given Fc variant for the different classes of FcγRs significantly affects the capacity of an Fc variant to target a given antigen for treatment of a given disease, the specificity or selectivity of an Fc variant for different polymorphic forms of these receptors may in part determine which research or pre-clinical experiments may be appropriate for testing, and ultimately which patient populations may or may not respond to treatment. Thus, the specificity or selectivity of Fc variants disclosed herein to Fc receptor polymorphisms, including but not limited to FcγRIIa, FcγRIIIa, and the like, may be used to guide the selection of valid research and pre-clinical experiments, clinical trial design, patient selection, dosing dependence, and/or other aspects concerning clinical trials.

Fc variants disclosed herein may comprise modifications that modulate interaction with Fc receptors other than FcγRs, including but not limited to complement proteins, FcRn, and Fc receptor homologs (FcRHs). FcRHs include but are not limited to FcRFH, FcRH2, FcRH3, FcRH4, FcRH5, and FcRH6 (Davis et al., 2002, Immunol. Reviews 190:123-136).

An important parameter that determines the most beneficial selectivity of a given Fc variant to treat a given disease is the context of the Fc variant. Thus, the Fc receptor selectivity or specificity of a given Fc variant will provide different properties depending on whether it composes an antibody, Fc fusion, or Fc variants with a coupled fusion partner. In one embodiment, an Fc receptor specificity of the Fc variant disclosed herein will determine its therapeutic utility. The utility of a given Fc variant for therapeutic purposes will depend on the epitope or form of the target antigen and the disease or indication being treated. For some targets and indications, greater FcγRIIb affinity and reduced activating FcγR-mediated effector functions may be beneficial. For other target antigens and therapeutic applications, it may be beneficial to increase affinity for FcγRIIb, or increase affinity for both FcγRIIb and activating receptors.

### Formulation and Pharmaceutical Compositions

The present invention provides pharmaceutical compositions and formulations of obexelimab. The pharmaceutical compositions, formulations and related methods of the invention are useful for delivering obexelimab and for the treatment of SLE. Formulations of obexelimab disclosed herein are prepared for storage by mixing said antibody having the desired degree of purity with optional pharmaceutically acceptable carriers, excipients or stabilizers (Remington's Pharmaceutical Sciences 16^{th} edition, Osol, A. Ed., 1980, incorporated entirely by reference), in the form of lyophilized formulations or aqueous solutions.

In some embodiments, pharmaceutical compositions of interest comprise obexelimab at various concentrations. In some embodiments, suitable formulations may comprise obexclimab at a concentration up to about 250 mg/ml (e.g., up to about 225 mg/ml, up to 200 mg/ml, up to 150 mg/ml, up to 140 mg/ml, up to 130 mg/ml, up to 125 mg/ml, up to 120 mg/ml, up to 115 mg/ml, up to 110 mg/ml, up to 105 mg/ml, up to 100 mg/ml, up to 90 mg/ml, up to 80 mg/ml, up to 70 mg/ml, up to 60 mg/ml, up to 50 mg/ml, up to 40 mg/ml, up to 30 mg/ml, up to 25 mg/ml, up to 20 mg/ml, up to 10 mg/ml). In some embodiments, suitable formulations may comprise obexelimab at a concentration up to about 250 mg/ml. In some embodiments, suitable formulations may comprise obexelimab at a concentration up to about 225 mg/ml. In some embodiments, suitable formulations may comprise obexelimab at a concentration up to about 200 mg/ml. In some embodiments, suitable formulations may comprise obexelimab at a concentration up to about 150 mg/ml. In some embodiments, suitable formulations may comprise obexclimab at a concentration up to about 140 mg/ml. In some embodiments, suitable formulations may comprise obexclimab at a concentration up to about 130 mg/ml. In some embodiments, suitable formulations may comprise obexelimab at a concentration up to about 125 mg/ml. In some embodiments, suitable formulations may comprise obexelimab at a concentration up to about 120 mg/ml. In some embodiments, suitable formulations may comprise obexelimab at a concentration up to about 115 mg/ml. In some embodiments, suitable formulations may comprise obexelimab at a concentration up to about 110 mg/ml. In some embodiments, suitable formulations may comprise obexelimab at a concentration up to about 105 mg/ml. In some embodiments, suitable formulations may comprise obexelimab at a concentration up to about 100 mg/ml. In some embodiments, suitable formulations may comprise obexelimab at a concentration up to about 90 mg/ml. In some embodiments, suitable formulations may comprise obexelimab at a concentration up to about 80 mg/ml. In some embodiments, suitable formulations may comprise obexelimab at a concentration up to about 70 mg/ml. In some embodiments, suitable formulations may comprise obexelimab at a concentration up to about 60 mg/ml. In some embodiments, suitable formulations may comprise obexelimab at a concentration up to about 50 mg/ml. In some embodiments, suitable formulations may comprise obexclimab at a concentration up to about 40 mg/ml. In some embodiments, suitable formulations may comprise obexelimab at a concentration up to about 30 mg/ml. In some embodiments, suitable formulations may comprise obexelimab at a concentration up to about 25 mg/ml. In some embodiments, suitable formulations may comprise obexclimab at a concentration up to about 20 mg/ml. In some embodiments, suitable formulations may comprise obexelimab at a concentration up to about 10 mg/ml.

In some embodiments, suitable formulations may contain obexelimab at a concentration ranging between about 10-300 mg/ml (e.g., about 10-250 mg/ml, about 10-200 mg/ml, about 10-180 mg/ml, about 10-160 mg/ml, about 10-150 mg/ml, about 10-140 mg/ml, about 10-130 mg/ml, about 10-125 mg/ml, about 100-125 mg/ml, about 100-180 mg/ml, about 100-150 mg/ml, about 100-130 mg/ml, about 100-125 mg/ml, about 100-170 mg/ml, about 100-160 mg/ml, about 100-150 mg/ml, about 100-200 mg/ml, about 120-130 mg/ml). In some embodiments, suitable formulations may contain obexelimab at a concentration ranging between about 10-300 mg/ml. In some embodiments, suitable formulations may contain obexelimab at a concentration ranging between about 10-250 mg/ml. In some embodiments, suitable formulations may contain obexclimab at a concentration ranging between about 10-200 mg/ml. In some embodiments, suitable formulations may contain obexelimab at a concentration ranging between about 10-180 mg/ml. In some embodiments, suitable formulations may contain obexclimab at a concentration ranging between about 10-160 mg/ml. In some embodiments, suitable formulations may contain obexelimab at a concentration ranging between about 10-150 mg/ml. In some embodiments, suitable formulations may contain obexclimab at a concentration ranging between about 10-140 mg/ml. In some embodiments, suitable formulations may contain obexelimab at a concentration ranging between about 10-130 mg/ml. In some embodiments, suitable formulations may contain obexclimab at a concentration ranging between about 10-125 mg/ml. In some embodiments, suitable formulations may contain obexelimab at a concentration ranging between about 100-125 mg/ml. In some embodiments, suitable formulations may contain obexelimab at a concentration ranging between about 100-180 mg/ml. In some embodiments, suitable formulations may contain obexelimab at a concentration ranging between about 100-150 mg/ml. In some embodiments, suitable formulations may contain obexclimab at a concentration ranging between about 100-130 mg/ml. In some embodiments, suitable formulations may contain obexelimab at a concentration ranging between about 100-125 mg/ml. In some embodiments, suitable formulations may contain obexclimab at a concentration ranging between about 100-170 mg/ml. In some embodiments, suitable formulations may contain obexclimab at a concentration ranging between about 100-160 mg/ml. In some embodiments, suitable formulations may contain obexelimab at a concentration ranging between about 100-150 mg/ml. In some embodiments, suitable formulations may contain obexelimab at a concentration ranging between about 100-200 mg/ml. In some embodiments, suitable formulations may contain obexelimab at a concentration ranging between about 120-130 mg/ml.

In some embodiments, formulations suitable for administration may contain obexelimab at a concentration of approximately 100 mg/ml, 115 mg/ml, 120 mg/ml, 125 mg/ml, 130 mg/ml, 135 mg/ml, 140 mg/ml, 145 mg/ml, 150 mg/ml, 200 mg/ml or 300 mg/ml. In some embodiments, formulations suitable for administration may contain obexelimab at a concentration of approximately 100 mg/ml. In some embodiments, formulations suitable for administration may contain obexclimab at a concentration of approximately 115 mg/ml. In some embodiments, formulations suitable for administration may contain obexelimab at a concentration of approximately 120 mg/ml. In some embodiments, formulations suitable for administration may contain obexelimab at a concentration of approximately 125 mg/ml. In some embodiments, formulations suitable for administration may contain obexelimab at a concentration of approximately 130 mg/ml. In some embodiments, formulations suitable for administration may contain obexelimab at a concentration of approximately 135 mg/ml. In some embodiments, formulations suitable for administration may contain obexelimab at a concentration of approximately 140 mg/ml. In some embodiments, formulations suitable for administration may contain obexelimab at a concentration of approximately 145 mg/ml. In some embodiments, formulations suitable for administration may contain obexelimab at a concentration of approximately 150 mg/ml. In some embodiments, formulations suitable for administration may contain obexelimab at a concentration of approximately 200 mg/ml. In some embodiments, formulations suitable for administration may contain obexelimab at a concentration of approximately 300 mg/ml.

In some embodiments, isotonic solutions are used. In some embodiments, slightly hypertonic solutions (e.g., up to 300 mM (e.g., up to 250 mM, 200 mM, 175 mM, 150 mM, 125 mM) sodium chloride in 5 mM sodium phosphate at pH 7.0) and sugar-containing solutions (e.g., up to 3% (e.g., up to 2.4%, 2.0%, 1.5%, 1.0%) sucrose in 5 mM sodium phosphate at pH 7.0). In some embodiments, a suitable formulation composition is saline (e.g., 150 mM NaCl in water).

Many therapeutic agents, and in particular the antibodies of the present invention, require controlled pH and specific excipients to maintain their solubility and stability in the pharmaceutical compositions of the present invention.

The pH of the pharmaceutical composition is an additional factor which is capable of altering the solubility of obexelimab in an aqueous pharmaceutical composition. In some embodiments, pharmaceutical compositions of the present invention contain one or more buffers. In some embodiments, compositions according to the invention contain an amount of buffer sufficient to maintain the optimal pH of said composition between about 4.0-8.0, between about 5.0-7.5, between about 5.5-7.0, between about 6.0-7.0 and between about 6.0-7.5. In other embodiments, the buffer comprises up to about 50 mM (e.g., up to about 45 mM, 40 mM, 35 mM, 30 mM, 25 mM, 20 mM, 15 mM, 10 mM, 5 mM) of sodium phosphate. Suitable buffers include, for example acetate, succinate, citrate, phosphate, other organic acids and tris (hydroxymethyl) aminomethane (“Tris”).

Suitable buffer concentrations can be from about 1 mM to about 100 mM, or from about 3 mM to about 20 mM, depending, for example, on the buffer and the desired isotonicity of the formulation. In some embodiments, a suitable buffering agent is present at a concentration of approximately 1 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, formulations contain an isotonicity agent to keep the formulations isotonic. Exemplary isotonicity agents include, but are not limited to, glycine, sorbitol, mannitol, sodium chloride and arginine. In some embodiments, suitable isotonic agents may be present in formulations at a concentration from about 0.01-5% (e.g., 0.05, 0.1, 0.15, 0.2, 0.3, 0.4, 0.5, 0.75, 1.0, 1.25, 1.5, 2.0, 2.5, 3.0, 4.0 or 5.0%) by weight.

In some embodiments, formulations may contain a stabilizing agent to protect the antibody. Typically, a suitable stabilizing agent is a non-reducing sugar such as sucrose, raffinose, trehalose, or amino acids such as glycine, arginine and methionine. The amount of stabilizing agent in a formulation is generally such that the formulation will be isotonic. However, hypertonic formulations may also be suitable. In addition, the amount of stabilizing agent must not be too low such that an unacceptable amount of degradation/aggregation of the antibody occurs. Exemplary stabilizing agent concentrations in the formulation may range from about 1 mM to about 400 mM (e.g., from about 30 mM to about 300 mM, and from about 50 mM to about 100 mM), or alternatively, from 0.1% to 15% (e.g., from 1% to 10%, from 5% to 15%, from 5% to 10%) by weight. In some embodiments, the ratio of the mass amount of the stabilizing agent and the therapeutic agent is about 1:1. In other embodiments, the ratio of the mass amount of the stabilizing agent and the therapeutic agent can be about 0.1:1, 0.2:1, 0.25:1, 0.4:1, 0.5:1, 1:1, 2:1, 2.6:1, 3:1, 4:1, 5:1, 10:1, or 20:1. In some embodiments, suitable for lyophilization, the stabilizing agent is also a lyoprotectants.

In some embodiments, it is desirable to add a surfactant to formulations. Exemplary surfactants include nonionic surfactants such as Polysorbates (e.g., Polysorbates 20 or 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linolcamidopropyl-, myristamidopropyl-, palmidopropyl-, or isostearamidopropyl-betaine (e.g., lauroamidopropyl); myristarnidopropyl-, palmidopropyl-, or isostearamidopropyl-dimethylamine; sodium methyl cocoyl-, or disodium methyl ofcyl-taurate; and the MONAQUAT™ series (Mona Industries, Inc., Paterson, N.J.), polyethyl glycol, polypropyl glycol, and copolymers of ethylene and propylene glycol (e.g., Pluronics, PF68, etc). Typically, the amount of surfactant added is such that it reduces aggregation of the protein and minimizes the formation of particulates or effervescences. For example, a surfactant may be present in a formulation at a concentration from about 0.001-0.5% (e.g., about 0.005-0.05%, or 0.005-0.01%). In particular, a surfactant may be present in a formulation at a concentration of approximately 0.005%, 0.01%, 0.02%, 0.1%, 0.2%, 0.3%, 0.4%, or 0.5%, etc.

In some embodiments, suitable formulations may further include one or more bulking agents, in particular, for lyophilized formylations. A “bulking agent” is a compound which adds mass to the lyophilized mixture and contributes to the physical structure of the lyophilized cake. For example, a bulking agent may improve the appearance of lyophilized cake (e.g., essentially uniform lyophilized cake). Suitable bulking agents include, but are not limited to, sodium chloride, lactose, mannitol, glycine, sucrose, trehalose, hydroxyethyl starch. Exemplary concentrations of bulking agents are from about 1% to about 10% (e.g., 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, and 10.0%).

Formulations in accordance with the present invention can be assessed based on product quality analysis, reconstitution time (if lyophilized), quality of reconstitution (if lyophilized), high molecular weight, moisture, and glass transition temperature. Typically, protein quality and product analysis include product degradation rate analysis using methods including, but not limited to, size exclusion HPLC (SE-HPLC), cation exchange-HPLC (CEX-HPLC), X-ray diffraction (XRD), modulated differential scanning calorimetry (mDSC), reversed phase HPLC (RP-HPLC), multi-angle light scattering (MALS), fluorescence, ultraviolet absorption, nephelometry, capillary electrophoresis (CE), SDS-PAGE, and combinations thereof. In some embodiments, evaluation of product in accordance with the present invention may include a step of evaluating appearance (either liquid or cake appearance).

Generally, formulations (lyophilized or aqueous) can be stored for extended periods of time at room temperature. Storage temperature may typically range from 0° C. to 45° C. (e.g., 4° C., 20° C., 25° C., 45° C., etc.). Formulations may be stored for a period of months to a period of years. Storage time generally will be 24 months, 12 months, 6 months, 4.5 months, 3 months, 2 months or 1 month. Formulations can be stored directly in the container used for administration, eliminating transfer steps.

Formulations can be stored directly in the lyophilization container (if lyophilized), which may also function as the reconstitution vessel, eliminating transfer steps. Alternatively, lyophilized product formulations may be measured into smaller increments for storage. Storage should generally avoid circumstances that lead to degradation of the proteins, including but not limited to exposure to sunlight, UV radiation, other forms of electromagnetic radiation, excessive heat or cold, rapid thermal shock, and mechanical shock.

In some embodiments, formulations according to the present invention are in a liquid or aqueous form. In some embodiments, formulations of the present invention are lyophilized. Such lyophilized formulations may be reconstituted by adding one or more diluents thereto prior to administration to a patient. Suitable diluents include, but are not limited to, sterile water, bacteriostatic water for injection and sterile saline solution. Preferably, upon reconstitution, the antibody contained therein is stable, soluble and demonstrates tolerability upon administration to a patient.

The pharmaceutical compositions of the present invention are characterized by their tolerability. As used herein, the terms “tolerable” and “tolerability” refer to the ability of the pharmaceutical compositions of the present invention to not elicit an adverse reaction in the patient to whom such composition is administered, or alternatively not to elicit a serious adverse reaction in the patient to whom such composition is administered. In some embodiments, the pharmaceutical compositions of the present invention are well tolerated by the patient to whom such compositions are administered.

Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition that comprises the antibody disclosed herein may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Some embodiments include at least one of the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The formulations to be used for in vivo administration may be sterile. In some embodiments, the formulation is sterilized by filtration through sterile filtration membranes.

Nonlimiting examples of buffers include phosphate, citrate, acetate, glutamate, carbonate, tartrate, triethanolamine (TRIS), glycylglycine, histidine, glycine, lysine, arginine, and other organic acids. More specifically, non-limiting examples of buffers include HEPES sodium, MES, potassium phosphate, potassium thiocyanate, sterilant, TAE, TBE, ammonium sulfate/HEPES, BuffAR, sodium acetate, sodium carbonate, sodium citrate, sodium dihydrogen phosphate, disodium hydrogen phosphate, and sodium phosphate. Additionally, the buffer may be various hydrate forms. For example, the buffer may be a monohydrate, a dihydrate, a trihydrate, a tetrahydate, a pentahydrate, a hexahydrate, a heptahydrate, an octahydrate, a nonahydrate, a decahydrate, an undecahydrate, and a dodecahydrate. Occasionally, a hydrate may be fractional such as a hemihydrate or a sequihydrate. Nonlimiting examples of tonicity modifies include sodium chloride, acetic acid, L-proline, dextrose, mannitol, potassium chloride, glycerin, and glycerol. Non-limiting example of solvents include water, propylene glycol, polyethylene glycols, ethanol, dimethyl sulfoxide, N-methyl-2-pyrrolidone, glycofurol, Solketal™, glycerol formal, acetone, tetrahydrofurfuryl alcohol, diglyme, dimethyl isosorbide, and ethyl lactate. Non-limiting examples of solvents include polysorbates (e.g., polysorbate-20, polysorbate-80), polyoxyethylene sorbitan monooleate (Tween 80), sorbitan monooleate polyoxyethylene sorbitan monolaurate (Tween 20), sorbitan trioleate (span 85), lecithin, and polyoxyethylene polyoxypropylene copolymers (Pluronics, Pluronic F-68).

The amounts of obexelimab, buffer, tonicity modifier, solvent, and surfactants may vary. In some embodiments, obexelimab is formulated at a concentration of 125 mg/mL obexclimab, 2.35 mg/mL sodium acetate trihydrate, 0.17 mg/mL acetic acid (at density 1.053 g/mL), 30 mg/mL L-proline, 0.1 mg/mL polysorbate 80, pH 5.5. In some embodiments, obexclimab is formulated at a concentration of 80-200 mg/mL obexelimab, 1.5-3 mg/mL sodium acetate trihydrate, 0. 1-0.2 mg/mL acetic acid (at density 1.053 g/mL), 10-50 mg/mL L-proline, 0.05-0.2 mg/mL polysorbate 80, pH 5.0-6.0. In some embodiments, obexelimab is formulated at a concentration of 122-127 mg/mL obexelimab, 2.0-2.5 mg/mL sodium acetate trihydrate, 0.15-0.19 mg/mL acetic acid (at density 1.053 g/mL), 25-35 mg/mL L-proline, 0.05-0.15 mg/mL polysorbate 80, pH 5.0-6.0.

In certain embodiments, formulation comprises obexelimab, one or more buffers, one or more tonicity modifiers, one or more solvents, and one or more surfactants. In some embodiments, the buffer can be a sodium acetate buffer. For example, the buffer can be sodium acetate trihydrate. In some embodiments, the tonicity modifier can be acetic acid, L-proline, and combinations thereof. In another embodiment, the solvent is water.

In some embodiments, the surfactant is a polysorbate. In some embodiments, the polysorbate is polysorbate-80. In some embodiments, a formulation comprises obexelimab, sodium acetate trihydrate, acetic acid and L-proline, water, and polysorbate-80.

In some embodiments, the formulation comprises obexelimab in an amount from about 1 mg to about 500 mg per mL or about 50 mg to about 250 mg per mL or about 100 mg to about 250 mg per mL, sodium acetate trihydrate in an amount from about 1 to about 1 0 mg per mL or about 1 to about 5 mg per mL or about 1 to about 2.5 mg per mL, acetic acid and L-proline in an amount from about 5 to about 50 mg per mL or about 10 to about 50 mg per mL or about 20 to about 40 mg per mL, water up to about 1 mL, and polysorbate-80 in an amount from about 0.01 mg to about 1 mg per mL or about 0.01 to about 0.5 mg/ml or about 0.05 to about 0.2 mg/ml. Specifically, a formulation comprises obexelimab in an amount from about 100 mg to about 250 mg/ml, sodium acetate trihydrate in an amount from about 1 to about 2.5 mg/ml, acetic acid and L-proline in an amount from about 20 to about 40 mg/ml, water up to about 1 mg/ml, and polysorbate-80 in an amount from about 0.05 to about 0.2 mg per ml

Obexelimab as disclosed herein may also be formulated as immunoliposomes. A liposome is a small vesicle comprising various types of lipids, phospholipids and/or surfactant that is useful for delivery of obexelimab to a mammal. Liposomes containing obexelimab are prepared by methods known in the art. Liposomes with enhanced circulation time are disclosed in U.S. Pat. No. 5,013,556, incorporated entirely by reference. In some embodiments, obexelimab is formulated in liposomes generated by the reverse phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

In some embodiments, obexelimab is entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980, incorporated entirely by reference.

In some embodiments, obexelimab sustained-release preparations may be prepared. Suitable examples of sustained-release preparations include semipermeable matrices of solid hydrophobic polymer, which matrices are in the form of shaped articles, e.g., films, or microcapsules. Examples of sustained-release matrices include polyesters, hydrogels (for example poly(2-hydroxyethyl-methacrylate), or poly(vinylalcohol)), polylactides (U.S. Pat. No. 3,773,919, incorporated entirely by reference), copolymers of L-glutamic acid and gamma ethyl-L-glutamate, non-degradable ethylene-vinyl acetate, degradable lactic acid-glycolic acid copolymers such as the Lupron Depot® (which are injectable microspheres composed of lactic acid-glycolic acid copolymer and leuprolide acetate), poly-D-(−)-3-hydroxybutyric acid, and ProLease® (commercially available from Alkermes), which is a microsphere-based delivery system composed of the desired bioactive molecule incorporated into a matrix of poly-DL-lactide-co-glycolide (PLG).

In some embodiments, the liquid pharmaceutical composition comprises 122-127 mg/mL obexclimab, 2.0-2.5 mg/mL sodium acetate trihydrate, at a pH 5.0-6.0, 0.15-0.19 mg/mL acetic acid (at density 1.053 g/mL), 25-35 mg/mL L-proline, 0.05-0.15 mg/mL polysorbate 80.

In some embodiments, the liquid pharmaceutical composition comprises about 125 mg/mL obexclimab, 2.35 mg/mL sodium acetate trihydrate, 0.17 mg/mL acetic acid, 30 mg/mL L-proline, 0.1 mg/mL polysorbate 80 at pH 5.5.

Treatment of SLE with Obexelimab

Provided herein are methods of treating SLE comprising administering obexelimab. In one aspect, the present invention provides a method of treating SLE, comprising administering obexelimab to a human patient at a dose of 250 mg once a week or an equivalent dosing regimen thereof. In some embodiments, the method comprises administering obexelimab to a human patient at a dose of 250 mg once a week.

In some embodiments, the patient meets the 2019 European League Against Rheumatism (EULAR) and the American College of Rheumatology (ACR) Classification Criteria for SLE. In some embodiments, the patient has accumulated ≥10 points according to the 2019 European League Against Rheumatism (EULAR) and the American College of Rheumatology (ACR) Classification Criteria for SLE. In some embodiments, the patient has moderate SLE. In some embodiments, the patient has severe SLE. In some embodiments, the patient has active SLE. In some embodiments, the disease activity score is greater than or equal to 6 according to hSLEDAI. In some embodiments, the disease activity score is greater than or equal to 7 according to hSLEDAI. In some embodiments, the disease activity score is greater than or equal to 8 according to hSLEDAI. In some embodiments, the disease activity score is greater than or equal to 9 according to hSLEDAI. In some embodiments, the disease activity score is greater than or equal to 10 according to hSLEDAI. In some embodiments, the disease activity score is 6 to 9 according to hSLEDAI. In some embodiments, the active SLE has a British Isles Lupus Assessment Group (BILAG)-2004 grade A or B in greater than or equal to 1 organ system. In some embodiments, the active SLE has a disease activity score in greater than or equal to 6 according to hSLEDAI and/or a BILAG-2004 grade A or B in greater than or equal to 1 organ system.

In some embodiments, obexelimab is administered subcutaneously. In some embodiments, obexelimab is administered at a dose of 250 mg. In some embodiments, obexelimab is administered at a dose of about 100 mg to about 250 mg. In some embodiments, obexelimab is administered at a dose of about 100 mg. In some embodiments, obexelimab is administered at a dose of about 125 mg. In some embodiments, obexelimab is administered at a dose of about 150 mg. In some embodiments, obexelimab is administered at a dose of about 175 mg. In some embodiments, obexelimab is administered at a dose of about 200 mg. In some embodiments, obexelimab is administered at a dose of about 225 mg. In some embodiments, obexelimab is administered at a dose of about 250 mg.

In some embodiments, obexelimab is administered once a week. In some embodiments, obexelimab is administered less frequently than once a week. In some embodiments, obexclimab is administered once every other week. In some embodiments, obexelimab is administered as a single injection. In some embodiments, obexelimab is administered as two or more injections, for example, obexelimab is administered as 3 injections, or 4 injections or more. In some embodiments, obexelimab is administered more frequently than once a week. In some embodiments, the weekly dose may be injected at once or divided into two or more injections throughout a week. In some embodiments, obexelimab is administered subcutaneously at a dose of 250 mg per week, at a concentration of 125 mg/ml, as 2×1 ml injections. In some embodiments, obexelimab is administered subcutaneously at a dose of 250 mg per week, at a concentration of 125 mg/ml, as 1×2 ml injection.

In some embodiments, the method comprises administering obexelimab subcutaneously at a dose of 250 mg every 7 days. In some embodiments, obexclimab is administered in a liquid formulation as a single injection. In some embodiments, obexelimab is administered in a liquid formulation as a single injection for a total dose of 250 mg. In some embodiments, the method comprises administering obexclimab subcutaneously at a dose of 125 mg twice a week. In some embodiments, obexelimab is administered as 2×1 mL injections. In some embodiments, obexelimab is administered as 2×1 mL injections for a total dose of 250 mg. In some embodiments, obexelimab is administered in a liquid formulation comprising 125 mg/mL obexelimab as 4×0.5 mL injections. In some embodiments, obexelimab is administered in a liquid formulation comprising 125 mg/mL obexelimab as 4×0.5 mL injections for a total dose of 250 mg.

In some embodiments, obexelimab is administered at a dose of 20-40 mg daily, 25-40 mg daily, or 30-40 mg daily. In some embodiments, obexelimab is administered at a dose of 35 mg daily.

In some embodiments, obexelimab is administered at a dose of about 100 mg to about 250 mg weekly. In some embodiments, obexelimab is administered at a dose of about 100 mg to about 250 mg every other week. In some embodiments, obexelimab is administered at a dose of about 100 mg weekly. In some embodiments, obexelimab is administered at a dose of about 100 mg every other week. In some embodiments, obexelimab is administered at a dose of about 125 mg weekly. In some embodiments, obexelimab is administered at a dose of about 125 mg every other week. In some embodiments, obexelimab is administered at a dose of about 150 mg weekly. In some embodiments, obexelimab is administered at a dose of about 150 mg every other week. In some embodiments, obexelimab is administered at a dose of about 175 mg weekly. In some embodiments, obexelimab is administered at a dose of about 175 mg every other week. In some embodiments, obexelimab is administered at a dose of about 200 mg weekly. In some embodiments, obexelimab is administered at a dose of about 200 mg every other week. In some embodiments, obexclimab is administered at a dose of about 225 mg weekly. In some embodiments, obexelimab is administered at a dose of about 225 mg every other week. In some embodiments, obexelimab is administered at a dose of about 250 mg weekly. In some embodiments, obexelimab is administered at a dose of about 250 mg every other week.

In some embodiments, obexelimab is administered at a dose of 500 mg. In some embodiments, obexelimab is administered at a dose of 500 mg every two weeks. In some embodiments, every 2-week dose may be injected at once or divided into two or more injections. In some embodiments, the dose is divided into 2 injections, 3 injections, 4 injections, 5 injections, 6 injections or more injections. In some embodiments, obexelimab is administered at a dose of 1000 mg. In some embodiments, the patient receives 1000 mg of obexelimab per month. In some embodiments, the 1000 mg monthly dose may be injected at once or divided into 4 or more injections. In some embodiments, the dose is divided into 4 injections, 5 injections, 6 injections, 7 injections, 8 injections or more injections.

### Standard of Care Therapies

In some embodiments, the patient has previously received or is further administered a standard of care therapy. In some embodiments, the patient has previously received a standard of care therapy. In some embodiments, the patient is further administered a standard of care therapy. In some embodiments, the standard of care therapy comprises administering an immunosuppressant, a corticosteroid, or an antimalarial. In some embodiments, the standard of care therapy comprises administering an immunosuppressant and a corticosteroid. In some embodiments, the standard of care therapy comprises administering an immunosuppressant and an antimalarial. In some embodiments, the standard of care therapy comprises administering a corticosteroid and an antimalarial. In some embodiments, the standard of care therapy comprises administering an immunosuppressant, a corticosteroid, and an antimalarial.

In some embodiments, the standard of care therapy comprises administering an immunosuppressant. In some embodiments, the immunosuppressant comprises mycophenolate mofetil, mycophenolate sodium, azathioprine, 6-mercaptopurine, methotrexate, cyclosporine, tacrolimus, or voclosporin. In some embodiments, the immunosuppressant comprises voclosporin. In some embodiments, the patient is administered: (a) about 3 g/day of mycophenolate mofetil; (b) about 2160 mg/day of mycophenolate sodium; (c) about 200 mg/day of azathioprinc; (d) about 200 mg/day of 6-mercaptopurine; (c) about 25 mg/week of methotrexate; (f) about 2 mg/kg/day of cyclosporine; (g) about 3 mg/day of tacrolimus; or, (h) about 23.7 mg of voclosporin twice daily. In some embodiments, the patient is administered: (a) at least about 3 g/day of mycophenolate mofetil; (b) at least about 2160 mg/day of mycophenolate sodium; (c) at least about 200 mg/day of azathioprine; (d) at least about 200 mg/day of 6-mercaptopurine; (e) at least about 25 mg/week of methotrexate; (f) at least about 2 mg/kg/day of cyclosporine; (g) at least about 3 mg/day of tacrolimus; or, (h) at least about 23.7 mg of voclosporin twice daily.

In some embodiments, the standard of care therapy comprises administering a corticosteroid. In some embodiments, the corticosteroid comprises a glucocorticoid steroid. In some embodiments, the glucocorticoid steroid comprises prednisone, hydrocortisone, prednisolone, triamcinolone, methylprednisolone, dexamethasone, betamethasone, or cortisone acetate.

In some embodiments, the patient administered obexelimab is receiving corticosteroids. In some embodiments, the patient is administered up to about 20 mg/day prednisone. In some embodiments, the patient is administered up to about 15 mg/day prednisone. In some embodiments, the patient is administered up to about 12.5 mg/day prednisone. In some embodiments, the patient is administered up to about 10 mg/day prednisone. In some embodiments, the patient is administered up to about 7.5 mg/day prednisone. In some embodiments, the patient is administered up to about 5 mg/day prednisone. In some embodiments, the patient is administered up to about 20 mg/day prednisone-equivalent. In some embodiments, the patient is administered up to about 15 mg/day prednisone-equivalent. In some embodiments, the patient is administered up to about 12.5 mg/day prednisone-equivalent. In some embodiments, the patient is administered up to about 10 mg/day prednisone-equivalent. In some embodiments, the patient is administered up to about 7.5 mg/day prednisone-equivalent. In some embodiments, the patient is administered up to about 5 mg/day prednisone-equivalent.

While corticosteroids are effective in managing the symptoms of SLE, administration of corticosteroids has numerous potential side-effects. Prolonged use of corticosteroids can increase the risk and severity of side effects. Furthermore, abruptly halting administration of corticosteroids is also associated with withdrawal symptoms and the potential for an increased risk of flare. As such when corticosteroids are administered to the patient, the dosage is ideally tapered as safety allows to minimize detrimental effects to the patient. In some embodiments, the corticosteroid is tapered. In some embodiments, the corticosteroid is tapered prior to administration of obexelimab. In some embodiments, the dosage of corticosteroid is stable prior to administration of obexelimab. In some embodiments, administration of obexelimab allows a patient to more rapidly taper corticosteroids.

In some embodiments, the corticosteroid is tapered to a dose of less than or equal to ≤5 mg/day prednisone-equivalent. In some embodiments, the corticosteroid is tapered to a dose of less than or equal to ≤7.5 mg/day prednisone-equivalent. In some embodiments, the corticosteroid is tapered to a dose of less than or equal to ≤10 mg/day prednisone-equivalent. In some embodiments, the corticosteroid is tapered to a dose of less than or equal to ≤12.5 mg/day prednisone-equivalent. In some embodiments, the corticosteroid is tapered to a dose of less than or equal to ≤15 mg/day prednisone-equivalent.

In some embodiments, the corticosteroid is tapered within 4 to 12 weeks of administration of obexelimab. In some embodiments, the corticosteroid is tapered within 4 weeks of administration of obexelimab. In some embodiments, the corticosteroid is tapered within 5 weeks of administration of obexelimab. In some embodiments, the corticosteroid is tapered within 6 weeks of administration of obexelimab. In some embodiments, the corticosteroid is tapered within 7 weeks of administration of obexelimab. In some embodiments, the corticosteroid is tapered within 8 weeks of administration of obexelimab. In some embodiments, the corticosteroid is tapered within 9 weeks of administration of obexelimab. In some embodiments, the corticosteroid is tapered within 10 weeks of administration of obexelimab. In some embodiments, the corticosteroid is tapered within 11 weeks of administration of obexelimab. In some embodiments, the corticosteroid is tapered within 12 weeks of administration of obexelimab. In some embodiments, the corticosteroid is tapered within 13 weeks of administration of obexelimab. In some embodiments, the corticosteroid is tapered within 14 weeks of administration of obexelimab. In some embodiments, the corticosteroid is tapered within 15 weeks of administration of obexelimab. In some embodiments, the corticosteroid is tapered within 16 weeks of administration of obexelimab. In some embodiments, the corticosteroid is tapered within 17 weeks of administration of obexelimab. In some embodiments, the corticosteroid is tapered within 18 weeks of administration of obexelimab. In some embodiments, the corticosteroid is tapered within 19 weeks of administration of obexelimab. In some embodiments, the corticosteroid is tapered within 20 weeks of administration of obexelimab. In some embodiments, the corticosteroid is tapered within 21 weeks of administration of obexelimab. In some embodiments, the corticosteroid is tapered within 22 weeks of administration of obexelimab. In some embodiments, the corticosteroid is tapered within 23 weeks of administration of obexelimab. In some embodiments, the corticosteroid is tapered within 24 weeks of administration of obexclimab.

In some embodiments, the corticosteroid is tapered to a dose of ≤5 mg/day prednisone-equivalent within 4 to 12 weeks of administered of obexelimab. In some embodiments, the corticosteroid is tapered to a dose of ≤5 mg/day prednisone-equivalent within 4 weeks of administered of obexelimab. In some embodiments, the corticosteroid is tapered to a dose of ≤5 mg/day prednisone-equivalent within 5 weeks of administered of obexelimab. In some embodiments, the corticosteroid is tapered to a dose of ≤5 mg/day prednisone-equivalent within 6 weeks of administered of obexelimab. In some embodiments, the corticosteroid is tapered to a dose of ≤5 mg/day prednisone-equivalent within 7 weeks of administered of obexelimab. In some embodiments, the corticosteroid is tapered to a dose of ≤5 mg/day prednisone-equivalent within 8 weeks of administered of obexelimab. In some embodiments, the corticosteroid is tapered to a dose of ≤5 mg/day prednisone-equivalent within 9 weeks of administered of obexelimab. In some embodiments, the corticosteroid is tapered to a dose of ≤5 mg/day prednisone-equivalent within 10 weeks of administered of obexelimab. In some embodiments, the corticosteroid is tapered to a dose of ≤5 mg/day prednisone-equivalent within 11 weeks of administered of obexelimab. In some embodiments, the corticosteroid is tapered to a dose of ≤5 mg/day prednisone-equivalent within 12 weeks of administered of obexelimab. In some embodiments, the corticosteroid is tapered to a dose of ≤5 mg/day prednisone-equivalent within 13 weeks of administered of obexelimab. In some embodiments, the corticosteroid is tapered to a dose of ≤5 mg/day prednisone-equivalent within 14 weeks of administered of obexelimab. In some embodiments, the corticosteroid is tapered to a dose of ≤5 mg/day prednisone-equivalent within 15 weeks of administered of obexelimab. In some embodiments, the corticosteroid is tapered to a dose of ≤5 mg/day prednisone-equivalent within 16 weeks of administered of obexelimab. In some embodiments, the corticosteroid is tapered to a dose of ≤5 mg/day prednisone-equivalent within 17 weeks of administered of obexelimab. In some embodiments, the corticosteroid is tapered to a dose of ≤5 mg/day prednisone-equivalent within 18 weeks of administered of obexelimab. In some embodiments, the corticosteroid is tapered to a dose of ≤5 mg/day prednisone-equivalent within 19 weeks of administered of obexelimab. In some embodiments, the corticosteroid is tapered to a dose of ≤5 mg/day prednisone-equivalent within 20 weeks of administered of obexelimab. In some embodiments, the corticosteroid is tapered to a dose of ≤5 mg/day prednisone-equivalent within 21 weeks of administered of obexelimab. In some embodiments, the corticosteroid is tapered to a dose of ≤5 mg/day prednisone-equivalent within 22 weeks of administered of obexelimab. In some embodiments, the corticosteroid is tapered to a dose of ≤5 mg/day prednisone-equivalent within 23 weeks of administered of obexelimab. In some embodiments, the corticosteroid is tapered to a dose of ≤5 mg/day prednisone-equivalent within 24 weeks of administered of obexclimab.

In some embodiments, the standard of care therapy comprises administering an antimalarial. In some embodiments, the antimalarial comprises hydroxychloroquine, quinacrine, or chloroquine. In some embodiments, the patient is administered: (a) about 400 mg/day of hydroxychloroquine; (b) about 100 mg/day of quinacrine; or (c) about 250 mg/day of chloroquine. In some embodiments, the patient is administered: (a) at least about 400 mg/day of hydroxychloroquine; (b) at least about 100 mg/day of quinacrine; or (c) at least about 250 mg/day of chloroquine.

### Disease Activity Assessments

In some embodiments, the patient achieves one or more of the following: (a) a response according to the BILAG-Based Composite Lupus Assessment (BICLA); (b) a response according to the Systemic Lupus Erythematosus Responder Index 4 (SRI-4), Systemic Lupus Erythematosus Responder Index 6 (SRI-6), or Systemic Lupus Erythematosus Responder Index 8 (SRI-8); (c) Lupus Low Disease Activity State (LLDAS); (d) a ≥50% improvement from baseline in Cutaneous Lupus Erythematosus Disease Area and Severity Index Activity Score (CLASI-50 response); (e) a ≥50% decrease in joint count (Joint-50 response) as compared to baseline; or (f) a change from baseline in Functional Assessment of Chronic Illness Therapy (FACIT) Fatigue Score.

### British Lupus Assessment Group-2004 Index (BILAG-2004)

In some embodiments, BILAG-2004 is used to assess patients treated with obexelimab. The BILAG disease activity index evaluates SLE activity in 9 organ systems, using a separate alphabetic score (A to E) assigned to each organ system defined as follows, BILAG A: severe disease activity; BILAG B: moderate disease activity; BILAG C: stable mild disease; BILAG D: system previously affected but now inactive; BILAG E: system never involved. Scoring is based on the principle of physician's intention to treat. BILAG-2004 may be scored as described in Isenberg DA & Gordon C, Lupus. 2000; 9 (9): 651-654, the contents of which is incorporated by reference in its entirety. In some embodiments, BILAG-2004 is scored as described in Isenberg DA & Gordon C, Lupus. 2000; 9 (9): 651-654.

Hybrid Systemic Lupus Erythematosus Disease Activity Index (hSLEDAI)

In some embodiments, hSLEDAI is used to assess patients treated with obexelimab. hSLEDAI is a weighted index used to assess disease activity across 24 different disease descriptors in patients with SLE. Each descriptor is scored as being absent or present. hSLEDAI may be scored as described in Thanou A et al., Lupus Sci Med. (2014): 1 (1): e000005, the contents of which is incorporated by reference in its entirety. In some embodiments, hSLEDAI is scored as described in Thanou A et al., Lupus Sci Med. (2014): 1 (1): e000005. In some embodiments, hSLEDAI is identical to the SELENA-SLEDAI with the exception of scoring of proteinuria, which uses the same definition as in the SLEDAI-2K.

### British Lupus Assessment Group (BILAG)-Based Composite Lupus Assessment (BICLA) Response

In some embodiments, BICLA is used to assess patients treated with obexelimab. In some embodiments, a patient achieves a BICLA Response if the following criteria are met:
all BILAG-2004 Grade A at baseline improved to Grade B, C, or D, and all BILAG-2004 Grade B at baseline improved to Grade C or D;
no BILAG-2004 worsening in other organ systems, as defined by no new Grade A and no greater than 1 new Grade B compared to baseline;
no worsening in the hSLEDAI total score compared with baseline;
<0.3-point increase on 3-point Physician's Global Assessment (PGA)-visual analog scale (VAS); and
no use of protocol-specified prohibited medications or any treatments considered by the AC to potentially impact Week 24 assessment, and no premature discontinuation of study treatment.

### Systemic Lupus Erythematosus Responder Index 4 (SRI-4)

In some embodiments, SRI-4 is used to assess patients treated with obexelimab. In some embodiments, a patient achieves response according to SRI-4 if the following criteria are met:

reduction from baseline of ≥4 points in hSLEDAI
no new organ system affected as defined by no new organ system with BILAG-2004 Grade A and no more than 1 new BILAG-2004 Grade B compared to baseline.
no increase ≥0.3 points on 3-point PGA-VAS
no use of protocol-specified prohibited medications or any treatments considered by the AC to potentially impact Week 24 assessment, and no premature discontinuation of study treatment

### Lupus Low Disease Activity State (LLDAS)

In some embodiments, LLDAS is used to assess patients treated with obexclimab. In some embodiments, a patient achieves LLDAS if the following criteria are met: (a) SLEDAI-2K≤4, with no activity in major organ systems (renal, central nervous system, cardiopulmonary, vasculitis, or fever) and no hemolytic anemia or gastrointestinal activity; (b) no new features of lupus disease activity compared with the previous assessment; (c) SLEDAI PGA≤1; (d) current prednisolone (or equivalent) dose≤7.5 mg daily; (c) well-tolerated standard maintenance doses of immunosuppressive drugs; (f) no premature discontinuation of study treatment

In some embodiments, hSLEDAI is used when evaluating if a patient achieves LLDAS. In some embodiments, criteria (b) of LLDAS is defined as no increase in hSLEDAI or BILAG score compared to a prior visit. In some embodiments, criteria (e) is defined as no protocol prohibited SLE treatment. In some embodiments, a patient achieves LLDAS if the following criteria are met: (a) hSLEDAI≤4, with no activity in major organ systems (renal, central nervous system, cardiopulmonary, vasculitis, or fever) and no hemolytic anemia or gastrointestinal activity; (b) no increase in hSLEDAI or BILAG score compared to prior visit; (c) SLEDAI PGA≤1; (d) current prednisolone (or equivalent) dose≤7.5 mg daily; (c) no protocol prohibited SLE treatment; and (f) no premature discontinuation of study treatment.

### Cutaneous Lupus Erythematosus Disease Area and Severity Index Activity Score (CLASI)

In some embodiments, CLASI is used to assess patients treated with obexelimab. CLASI is a scoring system to assess and document the degree of skin involvement and disease activity in patients with cutaneous lupus. The CLASI generates 2 separate scores: 1) the CLASI-A score represents CLE activity, and 2) the CLASI-D score represents CLE damage. CLASI may be scored as described in Albrecht J. et al., J Invest. Dermatol. 2005; 125 (5): 889-894, the contents of which is incorporated by reference in its entirety. In some embodiments, CLASI is scored as described in Albrecht J. et al., J Invest. Dermatol. 2005; 125 (5): 889-894.

### Joint Count

In some embodiments, Joint Count is used to assess patients treated with obexelimab. In some embodiments, 28-Joint Count is used to assess patients treated with obexelimab. In some embodiments, the 28-Joint Count assesses joints for tenderness and for swelling. In some embodiments, the 28-Joint Count is the total number of joints that are tender and/or swollen. In some embodiments, the joints are selected from shoulders, elbows, wrists, metacarpophalangeal (MCP) joints, proximal interphalangeal (PIP) joints, and knees.

In some embodiments, Joint-50 response is used to assess patient treated with obexelimab. In some embodiments, a Joint-50 response is defined as a 50% reduction from baseline in Joint Count. In some embodiments, the patient achieves a ≥50% decrease in joint count (Joint-50 response) as compared to baseline.

### Functional Assessment of Chronic Illness Therapy (FACIT) Fatigue Scale

In some embodiments, FACIT Fatigue Scale is used to assess patients treated with obexelimab. FACIT-Fatigue questionnaire serves as a measure of self-reported fatigue and the impact of fatigue on a person's daily life, activities, and overall well-being. The change from baseline in FACIT-F score may be analyzed using a mixed model repeated measures (MMRM) approach with terms for baseline score, stratification factors, treatment, visit and treatment by visit interaction. In some embodiments, patients treated with obexelimab are asked to self-report levels of fatigue in the past 7 days.

### Time to Flare

In some embodiments, time to flare is used to assess patients treated with obexelimab. In some embodiments, time to flare is defined as a severe flare (≥1 BILAG-2004 A score marked as new or worse) or moderate flare (≥2 BILAG-2004 B scores marked as new or worse). In some embodiments, time to first flare is calculated as the time from administration of obexelimab to the date flare criteria are met. In some embodiments, time to severe flare is calculated as the time from administration of obexelimab to the time when ≥1 BILAG-2004 A score marked as new or worse.

### Examples

### Example 1: Administration of Obexelimab for Treating SLE

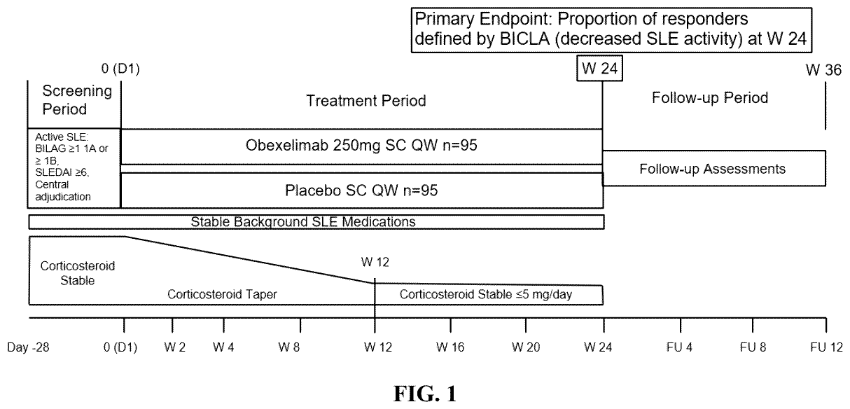

This Example describes an exemplary clinical trial for the administration of obexelimab to treat SLE. Approximately 190 patients are enrolled in a Phase 2, randomized, double-blind, placebo-controlled study to evaluate the efficacy and safety of obexelimab to reduce disease activity in patients with SLE. As shown in FIG. 1, patients with SLE are divided into an obexelimab treatment group and a placebo control group. The study consists of a Screening Period (Day-28 to Day-1) followed by a 24-week Treatment Period (Day 1 to Week 24) and a subsequent 12-week follow-up period (Week 24 to Week 36).

### Subject Selection

Patients enrolled in the study have active SLE at screening as defined by BILAG 2004 and hSLEDAI. Patients are scored on SLEDAI or BILAG at Screening and Day 1. When assessing post-baseline SLE disease activity, manifestations are scored as present at the visit if one of the following occurs: it is observed at the visit; it is not present at the current visit, but was present at visit 1 month prior and is reported by the patient to have continued after that visit; or, it is not present at the current visit, but was documented (e.g., photo or medical records) to have been present in the previous month. Patients are selected for the study based on the following inclusion and exclusion criteria.

### Inclusion Criteria

Patients are eligible to be included in the study only if all of the following criteria apply:
1. ≥18 to ≤70 years of age.
2. Capable of giving signed informed consent, which includes compliance with the requirements and restrictions listed in the ICF.
3. Diagnosed with SLE at least 24 weeks prior to Screening and meets the 2019 European League Against Rheumatism (EULAR)/American College of Rheumatology (ACR) classification criteria.
4. At Screening, at least one of the following:
   a) Antinuclear antibody (ANA) ≥1:80
   b) positive anti-dsDNA
   c) positive anti-Sm
5. Patient has all 3 of the following based on features active on the day of the visits:
   a) hSLEDAI ≥6 and clinical hSLEDAI ≥4 at Screening, and Clinical hSLEDAI ≥4 on Day 1.
      Note: Clinical points exclude laboratory tests, except proteinuria.
      Patients with proteinuria scored as present (i.e., urine protein/creatinine >500 mg/gm) at Screening are scored as present on Day 1 if a dipstick performed at the site on Day 1 is at least 2+ proteinuria. If the urine protein/creatinine >500 mg/g at Screening is not known to be stable based on recent past proteinuria testing, then a repeat urine protein/creatinine measurement is required during the Screening Period.
      b) BILAG-2004 grade A or B in ≥1 organ system at Screening and Day 1.
      c) In the opinion of the investigator and the adjudication committee, there is sufficient disease to warrant enrolment into a clinical trial with an investigational agent.
6. Patients must be treated with one or more of the following background nonbiologic lupus standard of care therapies: oral corticosteroid, antimalarial, and/or immunosuppressant.
   The treatment regimen must be as below:
      a) If taking oral corticosteroid: No increase in dosing regimen during the Screening Period, and at stable dose ≤20 mg/day prednisone-equivalent at least 2 weeks prior to Day 1.
      b) If taking antimalarial:
         No dose increase within 8 weeks prior to the Screening visit.
         After the Screening visit, dosing must be stable and as follows: hydroxychloroquine≤400 mg/day, quinacrine≤100 mg/day, chloroquine ≤250 mg/day.
      c) If taking immunosuppressant:
         No dose increase within 8 weeks prior to the Screening visit.
         After the Screening Visit, must be taking no more than one immunosuppressant, and at a stable dose as follows: Mycophenolate mofetil≤3 g/day, mycophenolate sodium≤2160 mg/day, azathioprine≤ 200 mg/day, 6-mercaptopurine≤100 mg/day, methotrexate≤25 mg/week, cyclosporine≤2 mg/kg/day, tacrolimus≤3 mg/day, voclosporin≤23.7 mg twice daily.
7. Female patient is eligible to participate if she is not pregnant, not breastfeeding, and at least 1 of the following conditions applies:
   a) Not a woman of childbearing potential (WOCBP).
   OR
   b) A WOCBP who meets all the following:
      Agrees to follow contraceptive guidance until at least 8 weeks after the last administration of IMP.
      Has a negative serum pregnancy test at screening and a negative urine pregnancy test at Day 1 prior to the first dose of study drug.
      Agrees to refrain from egg donation until at least 8 weeks after the last dose of IMP.
8. A male patient is eligible if the following conditions apply:
   a) Agrees to either sexual abstinence or use of contraception until at least 8 weeks after the last dose of IMP or is surgically sterile.
   AND
   b) Agree to refrain from donating sperm until at least 8 weeks after the last dose of IMP.

### Exclusion Criteria

Patients are excluded from the study if any of the following criteria apply:
1. Active lupus nephritis for which, in the opinion of the investigator or the adjudication committee, current medications are insufficient for patient's safety or additional therapy that is not permitted in the protocol is needed.
2. A history of thrombosis or embolism in the previous 6 months or any thrombotic history in the previous 12 months associated with antiphospholipid syndrome or another relevant hypercoagulable state. A thrombotic history associated with APS or another relevant hypercoagulable state more than 12 months prior to Screening visit is excluded if it is not treated per local standards with prophylactic anticoagulation.
3. Any active skin conditions other than cutaneous lupus erythematosus (CLE) that may interfere with the study assessment of CLE such as but not limited to psoriasis, dermatomyositis, systemic sclerosis.
4. Active severe neuropsychiatric or central nervous system (CNS) SLE.
5. Current inflammatory disease other than SLE (including, but not limited to, rheumatoid arthritis, psoriatic arthritis, spondyloarthropathy, reactive arthritis, scleroderma, dermatomyositis) that may interfere with the assessment of lupus signs and symptoms in the opinion of the investigator or central adjudicator. Current diagnosis of thyroiditis or secondary Sjogren's Syndrome is permitted.
6. Presence of uncontrolled or New York Heart Association class III or IV congestive heart failure.
7. Any condition or finding on physical exam, current or previous medical history, vital signs, 12-lead ECG, or laboratory tests that in the opinion of the investigator or central adjudicator might interfere with the evaluation of the investigational product or should otherwise exclude a patient.
8. Surgery (not considered minor by the investigator or the central adjudicator) within 4 weeks before Screening, or planned surgery during the study.
9. History of relevant allergies, including allergy to study drug or any murine or human-derived protein or immunoglobulin products, that in the opinion of the investigator or central adjudicator committee make inclusion in the study inappropriate.
10. History of any malignancy within 5 years, except for successfully treated nonmelanoma skin cancer or localized carcinoma in situ of the cervix.
11. History of drug abuse in the previous 12 months before Screening in the opinion of the investigator.
12. Use prior to Screening of one or more of the following:
   a) Within 6 months: rituximab or similar major B cell depleting biologic therapy, or T cell-depleting agent such as Campath (alemtuzumab).
   b) Within 1 month: belimumab or any inhibitor of BAFF and/or APRIL, anifrolumab, abatacept, infliximab, adalimumab, certolizumab, golimumab, etanercept, tocilizumab, anakinra, immunoglobulin, blood products, cyclophosphamide IV or oral, live or live-attenuated vaccine, other biologics with immunomodulating/immunosuppressive activity.
   c) Within 5 half-lives or 30 days, whichever is longer: Any investigational drug.
13. Use after the Screening Visit of medical treatment (including prescription drugs, non-prescription drugs, biological products, Chinese or herbal medicines, diet supplements, etc.) or health care products considered by the Investigator or central adjudicator to potentially impact the interpretation of study results.
14. Currently enrolled in another interventional clinical study.
15. Spot urine protein-creatinine ratio >3.0 mg/mg (339 mg/mmol), or estimated glomerular filtration rate <30 mL/min/1.73 m² calculated using the Chronic Kidney Disease Epidemiology Collaboration (CKD-EPI) equation, at Screening.
16. BMI <18 or >40.0 kg/m².
17. Acute hepatitis B infection (hepatitis B surface antigen-positive), active hepatitis C virus, or HIV infection. Patients will be excluded from the study if they have a positive test for active hepatitis B through detection of (a) hepatitis B surface antigen or (b) hepatitis B core antibody. In Japan and other locations where required by local regulations, patients are excluded if there is detection of (a) hepatitis B surface antigen, (b) hepatitis B surface antibody, or (c) hepatitis B core antibody.
18. Evidence of active tuberculosis (TB) or at high risk for TB as shown by at least one of the following:
   a. Documented history of active TB or latent TB, unless completion of treatment according to local guidelines.
   b. Positive, indeterminate, or invalid interferon-gamma release assay results at screening, unless treatment is documented. Patients with an indeterminate test result can repeat the test once either centrally or locally, but if the repeat test is also indeterminate, the patient is excluded.
   c. Signs or symptoms that could represent active TB.
   d. Chest radiograph, computed tomography, or magnetic resonance imaging that suggests possible diagnosis of TB.
19. Bacteria, mycobacteria, virus, systemic fungi, parasites, or opportunistic infection that are not resolved within 14 days before randomization. Urinary tract infection resolution must be confirmed with a negative urine culture.
20. Alanine amino-transferase (ALT) ≥2.5×ULN or aspartate amino-transferase (AST) ≥2.5×ULN; total bilirubin (TBIL) ≥1.5×ULN at Screening.
21. Total white blood cell (WBC) count <2,000/μL; Absolute neutrophil count (ANC) <1,500/μL; Platelet count <50,000/μL; if platelet count <120,000/μL at Screening Visit, then a repeat count during the Screening Period must be done unless the platelet level is documented to be stable or previously lower than the Screening value in the previous 2 months); Hemoglobin <9.0 g/dL, at Screening.
22. Employees or related personnel of the clinical research unit, the Sponsor, or Contract Research Organization.
23. History or evidence of a clinically unstable/uncontrolled disorder, medical condition (including, but not limited to, cardiopulmonary, oncologic, renal, hepatic, metabolic, hematologic, or psychiatric condition such as severe depression or suicidal ideations), that in the opinion of the investigator or adjudication committee, would pose a risk to patient safety or interfere with the study evaluation, procedures, or completion.
24. Any other reason that, in the opinion of the Investigator or adjudication committee, will make the patient not appropriate for enrollment, may cause undue risk to the patient, cause unacceptable risk of serious adverse event, prevent the patient from completing the study, or interfere with accurate clinical assessments.

### Administration of Obexelimab

On Day 1 of the study, patients are randomized 1:1 to receive obexelimab 250 mg subcutaneous (SC) once per week (QW) (e.g., two 1-mL 125 mg/mL injections) or placebo SC QW. Randomization is stratified based on disease activity (hSLEDAI 6-9 versus ≥10) and corticosteroid dose (prednisone equivalent) of <10 mg/day versus ≥10 mg/day. Background medications are not increased during the treatment period. Corticosteroids are tapered from a dose of ≤20 mg/day, prednisone equivalent. Attempts to taper corticosteroids are made at each study visit beginning at Week 4, until the target dose of ≤5 mg/day is reached. By the completion of the Week 12 study visit, it is envisioned that patients are on oral prednisone equivalent dose of ≤5 mg/day. All patients are evaluated at Week 2, Week 4, and then every 4 weeks thereafter until study completion for assessment and for glucocorticoid taper.

Table 2 shows an exemplary dosage regimen and the formulation of obexelimab.

TABLE 2

| | |
|---|---|
| Study Investigational | obexelimab |
| Product: | |
| Dosage formulation: | Solution for injection |
| Route of administration: | Subcutaneous |
| Formulation: | Formulation: 125 mg/mL obexelimab, 2.35 |
| | mg/mL sodium acetate trihydrate, 0.17 |
| | mg/mL acetic acid (at density 1.053 g/mL), |
| | 30 mg/mL L-proline, 0.1 mg/mL polysorbate |
| | 80, pH 5.5 |
| Unit dose | 125.0 (±10%) mg/mL |
| strength(s)/dosage | |
| level(s): | |
| Dosing instructions: | 2 × 1.0 mL administered SC to the |
| | abdominal region |
| Packaging: | Single-use 2-mL glass vials, each filled with |
| | 1.2 mL of drug product (e.g., a pre-filled |
| | syringe) |

During the study, patients are assessed for efficacy, safety, PK, PD, and immunogenicity at study visits. Assessments and procedures are conducted in the following order: (1) Patient-Reported Outcomes (PROs), (2) review of AEs/concomitant medications, (3) ECG and vital sign assessments, (4) physical exam and disease activity assessments, (5) blood draws, (6) study drug administration (if dose taken in clinic), and (7) instruct patients on medication and corticosteroid dosing. AEs/SAEs are collected from the signing of informed consent form throughout the study, and followed up until the event has resolved, has stabilized, or is otherwise explained, or until the patient is lost to follow-up. Patients avoid taking nonsteroidal anti-inflammatory drugs (NSAIDS) until after completion of all study assessments.

### Efficacy, Analyses, and Endpoints

The primary endpoint is the proportion of patients who achieve a response at Week 24, defined according to the BICLA Response. The proportion of patients with BICLA response are estimated for each treatment group, and the obexelimab group is compared to the placebo group using difference in proportions. Patients with missing data that prohibit the assessment of BICLA response are considered as non-responders. BICLA response is analyzed using a CMH test, stratified for baseline disease activity: hSLEDAI (6-9 vs ≥10) and corticosteroid dose (prednisone equivalent) (<10 mg/day vs ≥10 mg/day). The Mantel Haenszel stratum-weighted estimator of the risk difference is provided as the primary summary measure, along with the corresponding 90% CI. The common odds-ratio and corresponding 90% CI are also be presented.

Secondary endpoints are also used to evaluate the efficacy of obexelimab compared to placebo. Obexelimab efficacy is compared to placebo to: (1) reduce SLE disease activity, achieve low disease activity, and prevent flare; (2) achieve and maintain low corticosteroid dose; and (3) reduce fatigue. Secondary endpoints include:
Proportion of patients who achieve response according to the Systemic Lupus Erythematosus Responder Index 4 (SRI-4) at Week 24
Proportion of patients who achieve Lupus Low Disease Activity State (LLDAS) at Week 24
Time to flare
Proportion of patients achieving prednisone equivalent dose≤5 mg/day by the Week 12 study visit and maintained through Week 24 with no disease worsening
Proportion of patients who achieve a ≥50% improvement from baseline in Cutaneous Lupus Erythematosus Disease Area and Severity Index Activity Score (CLASI-50 response) at Week 24 compared to baseline. This is assessed among patients with a CLASI-A Score ≥8 at baseline
The proportion of patients who achieved ≥50% decrease in joint count (Joint-50 response) at Week 24 compared to baseline. This is assessed in patients with at least 4 active joints (tender and/or swollen in 28-joint count) at baseline
Change from Baseline in Functional Assessment of Chronic Illness Therapy (FACIT)-Fatigue Score at Week 24

Additional objectives of the study are the evaluation of obexelimab as compared to placebo for additional measures of efficacy, safety and tolerability, pharmacokinetics, pharmacodynamics, anti-drug antibodies, association of prespecified baseline biomarkers with response, and patient-reported outcomes. Safety endpoints will assess the number of patients with Treatment-Emergent Adverse Events, Serious Adverse Events, and injection-site reactions.

Additional endpoints related to efficacy include: proportion of patients who achieve a BICLA Response; proportion of patients who achieve both a BICLA Response and prednisone-equivalent corticosteroid dose≤5 mg/day; proportion of patients who achieve an SRI-4/6/8 Response; proportion of patients who achieve both a SRI-4/6/8 Response and prednisone-equivalent corticosteroid dose≤5 mg/day; time to onset of response (SRI-4/6/8, BICLA) sustained through Week 24; time to onset of response (SRI-4/6/8, BICLA) with prednisone-equivalent dose≤5 mg/day sustained through Week 24; hSLEDAI score; BILAG score; among patients seroactive at screening (defined as positive anti-double-strand DNA (dsDNA) and/or low complement [C3/C4]): BICLA response and SRI-4 response; proportion of patients who achieve Definition of Remission in SLE (DORIS); annualized flare rate; time to First BILAG-2004 severe flare; CLASI-A score; proportion of patients with baseline CLASI-A score ≥8 who achieve a CLASI-A score of ≤1, CLASI-20, -50, -70, or -90 Response; proportion of patients with baseline skin score on CLASI-A (i.e., scoring without mucous membrane or alopecia) ≥6 who have ≥50% decrease in score at Week 24; Joint count and Joint-50 response; change from baseline in Physician's Global Assessment (PGA)-visual analog scale (VAS) score; change in prednisone-equivalent corticosteroid dose compared to baseline; total prednisone-equivalent corticosteroid dose from Day 1 to Week 24; and, proportion of patients in pre-specified biomarker-defined subgroups with response (BICLA, SRI-4/6/8)

Additional endpoints related to safety include: numbers of patients with treatment-emergent adverse events (TEAEs), serious adverse events (SAEs), and injection site reactions

Additional endpoints related to pharmacokinetics include: serum concentration of obexclimab

Additional endpoints related to ADA include: anti-obexelimab antibodies

Additional endpoints related to pharmacodynamics include: absolute values and change from baseline in C3, C4, and anti-dsDNA; absolute values and change from baseline in immunoglobulin (Ig) subtypes (e.g., IgG, IgA, IgM); absolute levels and change in serum/plasma immune-mediated biomarkers (e.g., cytokines); whole blood immune cell (e.g., B cells) subset analysis and change; whole blood gene expression profiles (e.g., immune pathway modules, individual genes, etc.) and change; and, level of CD19+B cells bound by obexelimab (i.e., target receptor occupancy)

Additional endpoints related to patient-reported outcomes include: Patient's Global Impression of Fatigue Severity; Patient's Global Impression of Change in Fatigue Severity; Patient's Global Impression of SLE Symptom Severity; Patient's Global Impression of Change in SLE Symptom Severity; LupusQoL; FACIT-Fatigue score; and, Lupus Foundation of America Rapid Evaluation of Activity in Lupus (LFA-REAL) score, Patient Report.

Exploratory objectives of the study include evaluating the pharmacodynamic effects of obexelimab as compared to placebo on blood biomarker levels and the association of biomarkers and genetic polymorphisms with treatment response (i.e., predictive biomarkers). The associated exploratory endpoints include association of treatment response (efficacy, safety) with Serum cytokines/chemokines, whole blood gene expression biomarkers, serum SLE disease biomarkers (e.g., complement C3, C4, etc.), and blood immune cells (e.g., B cells) and genetic polymorphisms.

Descriptive analyses of the primary and secondary endpoints are conducted for the following subgroups:
baseline disease activity: hSLEDAI 6-9 vs ≥10
baseline corticosteroid dose (prednisone equivalent): <10 mg/day vs ≥10 mg/day
gender (male, female)
age (<65, ≥65 years)
ethnicity (Hispanic, not Hispanic)
race (White, Black, Asian, Other)
predictive Biomarker Subgroups
gene expression profile subsets
immune pathway modules
complement-associated biomarkers (e.g., C1q, C3, C4)
individual gene expression (e.g., CD27, APP)

### OTHER EMBODIMENTS

While a number of embodiments of this invention are described herein, the present disclosure and examples may be altered to provide other methods and compositions of this invention. Therefore, it will be appreciated that the scope of this invention is to be defined by the appended claims in addition to the specific embodiments that have been represented by way of example. All references cited herein are hereby incorporated by reference.

1. A method of treating systemic lupus erythematous (SLE), comprising administering obexelimab subcutaneously to a human patient with SLE at a dose of 250 mg once a week or an equivalent dosing regimen thereof.

2. The method of claim 1, wherein the patient meets the 2019 European League Against Rheumatism (EULAR) and the American College of Rheumatology (ACR) Classification Criteria for SLE.

3. The method of claim 1, wherein the patient:
a) is positive for antinuclear antibodies (ANA) at a titer of ≥1:80;
b) is positive for anti-Smith antibodies; or
c) is positive for anti-dsDNA antibodies
prior to the administration of obexelimab.

4. The method of claim 1, wherein the patient has active SLE.

5. The method of claim 4, wherein the active SLE:
a) has a disease activity score greater than or equal to 6 according to the hybrid Systemic Lupus Erythematosus Disease Activity Index (hSLEDAI); and/or,
b) has a British Isles Lupus Assessment Group (BILAG)-2004 grade A or B in greater than or equal to 1 organ system.

6. The method of claim 5, wherein the disease activity score is 6 to 9 according to hSLEDAI.

7. The method of claim 5, wherein the disease activity score is greater than or equal to 10 according to hSLEDAI.

8. The method of claim 1, wherein the patient has previously received or is further administered a standard of care therapy.

9. The method of claim 8, wherein the standard of care therapy comprises administering an immunosuppressant.

10. The method of claim 8, wherein the immunosuppressant comprises mycophenolate mofetil, mycophenolate sodium, azathioprine, 6-mercaptopurine, methotrexate, cyclosporine, tacrolimus, or voclosporin.

11. The method of claim 8, wherein the standard of care therapy comprises administering a corticosteroid.

12. The method of claim 11, wherein the corticosteroid comprises a glucocorticoid steroid.

13. The method of claim 12, wherein the glucocorticoid steroid comprises prednisone.

14. The method of claim 11, wherein the patient is administered up to about 20 mg/day prednisone-equivalent.

15. The method of claim 11, wherein the corticosteroid is tapered to a dose of less than or equal to 5 mg/day prednisone-equivalent within 12 weeks of administered of obexelimab.

16. The method of claim 8, wherein the standard of care therapy comprises administering an antimalarial.

17. The method of claim 8, wherein the antimalarial comprises hydroxychloroquine, quinacrine, or chloroquine.

18. The method of claim 1, wherein the patient achieves one or more of the following:
a) a response according to the BILAG-Based Composite Lupus Assessment (BICLA);
b) a response according to the Systemic Lupus Erythematosus Responder Index 4 (SRI-4), Systemic Lupus Erythematosus Responder Index 6 (SRI-6), or Systemic Lupus Erythematosus Responder Index 8 (SRI-8);
c) Lupus Low Disease Activity State (LLDAS);
d) a ≥50% improvement from baseline in Cutaneous Lupus Erythematosus Disease Area and Severity Index Activity Score (CLASI-50 response);
e) a ≥50% decrease in joint count (Joint-50 response) as compared to baseline; or
f) a change from baseline in Functional Assessment of Chronic Illness Therapy (FACIT) Fatigue Score.

19. The method of claim 1, wherein obexelimab is administered in a liquid formulation comprising 125 mg/mL obexelimab, 2.35 mg/mL sodium acetate trihydrate, 0.17 mg/mL acetic acid, 30 mg/mL L-proline, 0.1 mg/mL polysorbate 80 at pH 5.5.

20. (canceled)

21. The method of claim 19, wherein obexelimab is administered using a prefilled syringe or autoinjector.
